(19) United States
(12) Patent Application Publication
(10) Pub. No.: US 2026/0274768 A1
(43) Pub. Date: Sep. 17, 2026

(54) REDUCTION METHOD

(71) Applicant: NATIONAL UNIVERSITY CORPORATION HOKKAIDO UNIVERSITY, Sapporo-shi, Hokkaido (JP)

(72) Inventors: Hajime ITO, Sapporo-shi, Hokkaido (JP); Koji KUBOTA, Sapporo-shi, Hokkaido (JP)

(21) Appl. No.: 19/469,727

(22) PCT Filed: Mar. 29, 2024
(86) PCT No.: PCT/JP2024/013088

§ 371 Date: Sep. 26, 2025

(30) Foreign Application Priority Data
Apr. 7, 2023 (JP) 2023-062433

(51) Int. Cl. C07B 31/00

(52) U.S. Cl. CPC C07B 31/00

(57) ABSTRACT

A reduction method includes reducing a substrate (A) by mechanochemical treatment using at least a metal (B) and a proton source (C), wherein the substrate (A) is at least one compound including an unsaturated bond, and the metal (B) includes at least one selected from an alkali metal or an alkaline-earth metal. The method can provide a reaction product similar to a reaction product obtained by Birch reduction.

### TECHNICAL FIELD

The present invention relates to a reduction method including reducing a substrate (A) by mechanochemical treatment using at least a metal (B) and a proton source (C), wherein the substrate (A) includes at least one compound including an unsaturated bond, and the metal (B) includes at least one selected from an alkali metal or an alkaline-earth metal.

### BACKGROUND ART

In the synthesis of functional materials such as pharmaceuticals, liquid crystal compounds, organic electroluminescent compounds, battery materials, macromolecular compounds, oligomers, coloring materials, radiation-absorbing materials, information-recording materials, wavelength conversion materials, indicator materials, sensor materials, organic light-emitting diodes (OLED), and organic semiconductor materials, a reduction reaction of a compound including an unsaturated bond, such as a benzene ring, may be used.

NPL 1 discloses Birch reduction, in which a benzene ring is converted into a 1, 4-cyclohexadiene ring. In Birch reduction, a special reaction in which a stable benzene ring is partially reduced and selectively converted into a 1, 4-cyclohexadiene ring is carried out, and furthermore, an α, β-unsaturated carbonyl compound, a conjugated diene, and an alkyne are reduced. Birch reduction is one of the most basic reactions among many organic chemical reactions, and is a method that has been widely used for 50 years or more. On the other hand, Birch reduction uses liquid ammonia and an alkali metal and thus requires stringent reaction conditions including a cryogenic temperature and an inert atmosphere, and studies have been made to relax the reaction conditions.

NPL 2 discloses that Birch reduction is carried out in a solution system using lithium and an alkylamine.

NPL 3 discloses that Birch reduction is carried out in a solution system using lithium and ethylenediamine.

NPL 4 discloses that Birch reduction is carried out in a solution system using n-propylamine, ethylenediamine, and tertiary butanol as proton sources.

NPL 5 discloses that Birch reduction is carried out in a solution system using lithium, ethylenediamine, and an ether solvent such as tetrahydrofuran.

NPL 6 discloses a method in which lithium bis(trimethylsilyl)amide (LiHMDS) and a substrate are subjected to mechanochemical treatment to effect Birch reduction of the substrate.

PTL 1 discloses a method for reducing a compound including an unsaturated bond, the method including reacting a reducing agent with a compound including an unsaturated bond to reduce the unsaturated bond, the reducing agent including a solvated electron formed through dissolution of sodium in 1, 3-dimethyl-2-imidazolidinone in a mixture of a dispersion product obtained by dispersing the sodium in a dispersion solvent and the 1,3-dimethyl-2-imidazolidinone.

PTL 2 discloses a solid-phase organic reaction method including performing an organic reaction without a solvent by bringing metal particles at least some of which are nano-sized and a raw material into contact with each other in the presence of a solid dispersant, the metal particles serving as a reaction initiator.

### CITATION LIST

### Patent Literature

PTL 1: Japanese Unexamined Patent Application Publication No. 2019-194206
PTL 2: Japanese Unexamined Patent Application Publication No. 2012-20977

### Non Patent Literature

NPL 1: Journal of the Chemical Society, 1944, p. 430-436
NPL 2: Journal of the American Chemical Society, 1955, Vol. 77, No. 12, p. 3230-3233
NPL 3: The Journal of Organic Chemistry, 1957, Vol. 22, No. 8, p. 891-894
NPL 4: The Journal of Organic Chemistry, 2000, Vol. 65, No. 21, p. 7098-7104
NPL 5: Science, 2021, Vol. 374, Issue. 6568, p. 741-746
NPL 6: Chem, 2023, Vol. 3, Issue. 3, p. 576-591

### SUMMARY OF INVENTION

### Technical Problem

The reduction method (Birch reduction) disclosed in NPL 1 uses liquid ammonia, and thus requires strict temperature control at −33° C. or lower and an inert gas atmosphere and requires complicated experimental operations.

None of the reduction methods disclosed in NPLs 2 to 5 and PTL 1 use liquid ammonia, but each requires strict temperature control and an inert gas atmosphere and requires complicated experimental operations. Furthermore, since the reaction is carried out in a solution system, a certain amount of organic solvent is required, which may cause problems in terms of the working environment and safety of workers, global environmental protection, environmental load at the time of disposal of the used organic solvent, etc.

Organic synthesis reaction methods in which reaction raw materials are brought into direct contact with each other and no organic solvents are used are environmentally friendly, less problematic in terms of the working environment and safety, and academically and industrially interesting. As such an organic synthesis reaction method, a mechanochemical process has attracted attention. In the mechanochemical process, mechanical energy is applied to solid raw materials by means of grinding, shearing, impact, compression, or the like, so that the reaction can be carried out under solvent-free conditions or conditions where substantially no organic solvents are used. However, there has hitherto been no known method for reducing a substrate that is at least one compound including an unsaturated bond using the mechanochemical process and using a metal including at least one selected from an alkali metal or an alkaline-earth metal without using liquid ammonia.

An object of the present invention is to provide a reduction method including reducing a substrate that is at least one compound including an unsaturated bond by mechanochemical treatment using at least a metal including at least one selected from an alkali metal or an alkaline-earth metal and a proton source.

Another object of the present invention is to provide a reduction method that can provide a reaction product similar to the reaction product obtained by Birch reduction, wherein the reaction can be carried out under mild temperature conditions in an air atmosphere without using liquid ammonia, high activity is exhibited in the reduction reaction without a solvent or substantially without using an organic solvent, the reaction product can be obtained in a short time in high yield by a simple operation, and the reduction method is applicable to a wide variety of substrates.

### Solution to Problem

The present inventors have conducted intensive studies and found that the above objects can be achieved by a reduction method having specific means, thereby completing the present invention.

Thus, the present invention is as follows.

### [Item 1]

A reduction method including reducing a substrate (A) by mechanochemical treatment using at least a metal (B) and a proton source (C), wherein the substrate (A) is at least one compound including an unsaturated bond, and the metal (B) includes at least one selected from an alkali metal or an alkaline-earth metal.

### [Item 2]

The reduction method according to Item 1, wherein the substrate (A) includes at least one selected from the group consisting of an aromatic hydrocarbon compound, an aromatic heterocyclic compound, an internal alkyne-containing compound, and a conjugated alkene compound.

### [Item 3]

The reduction method according to Item 1 or 2, wherein the metal (B) includes at least one selected from the group consisting of lithium, sodium, and calcium.

### [Item 4]

The reduction method according to any one of Items 1 to 3, wherein the proton source (C) includes a sugar compound.

### [Item 5]

The reduction method according to any one of Items 1 to 4, wherein in the reducing by mechanochemical treatment, an organic solvent having a melting point of lower than 30° C. is used in an amount of 1.0 mL or less per mmol of the substrate.

### [Item 6]

The reduction method according to any one of Items 1 to 5, wherein the reducing by mechanochemical treatment is carried out under conditions of a temperature of 20° C. or higher and/or a non-inert atmosphere.

### Advantageous Effects of Invention

The present invention provides a reduction method including reducing a substrate that is at least one compound including an unsaturated bond by mechanochemical treatment using at least a metal including at least one selected from an alkali metal or an alkaline-earth metal and a proton source.

The present invention provides a reduction method that can provide a reaction product similar to the reaction product obtained by Birch reduction, wherein the reaction can be carried out under mild temperature conditions in an air atmosphere without using liquid ammonia, high activity is exhibited in the reduction reaction without a solvent or substantially without using an organic solvent, the reaction product can be obtained in a short time in high yield by simple means, and the reduction method is applicable to a wide variety of substrates.

### DESCRIPTION OF EMBODIMENTS

A reduction method according to the present invention is a reduction method including reducing a substrate (A) by mechanochemical treatment using at least a metal (B) and a proton source (C), wherein the substrate (A) is at least one compound including an unsaturated bond, and the metal (B) includes at least one selected from an alkali metal or an alkaline-earth metal. This will be described in detail below.

### [Substrate (A)]

The substrate (A) used in the reduction method according to the present invention is at least one compound including an unsaturated bond.

The reduction method according to the present invention is a method by which a reduction reaction that is substantially the same as widely known “Birch reduction” can be carried out. As the compound including an unsaturated bond, that is, the substrate (A), for example, at least one selected from the group consisting of an aromatic hydrocarbon compound, an aromatic heterocyclic compound, an internal alkyne-containing compound, a conjugated alkene compound, and an α, β-unsaturated carbonyl compound can be used.

### <Aromatic Hydrocarbon Compound>

In the reduction method according to the present invention, the aromatic hydrocarbon compound used as the substrate (A) is not particularly limited as long as it has at least one aromatic ring in its molecule. In the aromatic hydrocarbon compound, the number of carbon atoms forming the aromatic ring in the molecule is 6 or more and 30 or less, preferably 6 or more and 14 or less. The aromatic ring may constitute a fused ring or a spiro ring. The aromatic ring may be, for example, at least one selected from the group consisting of benzene, naphthalene, pentalene, indene, azulene, biphenylene, indacene, acenaphthylene, fluorene, phenalene, phenanthrene, anthracene, fluoranthene, aceanthrylene, triphenylene, pyrene, tetracene, perylene, tetraphenylene, pentacene, coronene, heptacene, and the like.

In the reduction method according to the present invention, one or more aromatic hydrocarbon compounds can be used.

In the aromatic hydrocarbon compound, the aromatic ring in the molecule may have one or more substituents at any positions. When there are a plurality of substituents, the substituents may be the same as or different from each other. Examples of the substituents include alkyl groups, aromatic groups, a hydroxyl group, alkoxy groups, a carboxyl group, carboxylic acid ester groups, acyl groups, and an amino group.

In the reduction method according to the present invention, the benzene ring in the aromatic hydrocarbon compound is usually reduced to a cyclohexadiene ring as shown below. In the reduction method according to the present invention, a compound resulting from further reduction of the cyclohexadiene ring is usually not preferentially produced, thus enabling a reaction that cannot be achieved by reduction by hydrogenation.

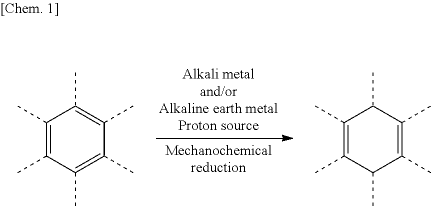

When a substituent is bonded to the aromatic ring, the regioselectivity of reduction depends on the nature of the substituent. In the reduction method according to the present invention, when the substituent is an electron-donating group (EDG) such as a hydroxyl group, an alkoxy group, a primary amino group, a secondary amino group, or an alkyl group, the reaction is as follows: hydrogen atoms are added to the 2— and 5-positions (2,5-dihydro type). The position of the electron-donating group on the 1, 4-cyclohexadiene ring is at a carbon atom constituting an unsaturated bond.

When the substituent is an electron-donating group, the rate of reduction is generally lower than that of an unsubstituted compound.

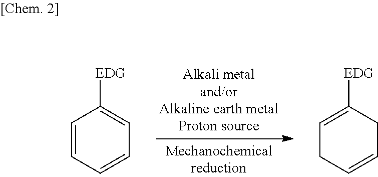

In the reduction method according to the present invention, when the substituent is an electron-withdrawing group (EWG) such as a carboxyl group, a phenyl group, an acyl group, or a carboxylic acid ester group, the reaction is as follows: hydrogen atoms are added to the 1- and 4-positions (1,4-dihydro type). The position of the electron-withdrawing group on the 1, 4-cyclohexadiene ring is at a carbon atom not constituting an unsaturated bond.

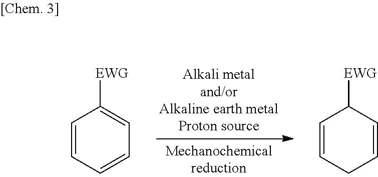

In the reduction method according to the present invention, when naphthalene was used as the substrate (A), 1, 2, 3, 4, 5, 8-hexahydronaphthalene was preferentially produced by a method described in Example A23 given later, and 1, 4, 5, 8-tetrahydronaphthalene was preferentially produced by a method described in B25 given later.

In the reduction method according to the present invention, when anthracene was used as the substrate (A), 9, 10-dihydroanthracene was preferentially produced as described in Examples A20 and B26 given later.

In the reduction method according to the present invention, when acenaphthylene was used as the substrate (A), acenaphthene was preferentially produced as described in Example A24 given later.

In the reduction method according to the present invention, when m-anisic acid (3-methoxybenzoic acid) was used as the substrate (A), 5-oxo-2-cyclohexene-1-carboxylic acid was preferentially produced as described in Example B42 given later.

In the reduction method according to the present invention, when 1-indanol was used as the substrate (A), 2, 3, 4, 7-tetrahydro-1H-indene was preferentially produced as described in Example B45 given later. In the reduction method according to the present invention, 4, 7-dihydro-1-indanol, a reaction product resulting from reduction of the phenyl ring of 1-indanol, was not preferentially produced.

In the reduction method according to the present invention, when N-(2-phenylethyl) acetamide was used as the substrate (A), 1-(2-aminoethyl)-1, 4-cyclohexadiene was preferentially produced as described in Example B46 given later. In the reduction method according to the present invention, N-[2-(1, 4-cyclohexadienyl)ethyl]acetamide, a reaction product resulting from reduction of the phenyl ring of N-(2-phenylethyl) acetamide, was not preferentially produced.

In the reduction method according to the present invention, when tetraphenylethylene (a compound in which an internal alkene group is bonded to aromatic rings) was used as the substrate (A), 1, 1, 2, 2-tetraphenylethane resulting from reduction of the internal alkene group alone was preferentially produced as described in Examples A25 and B47 given later.

In the reduction method according to the present invention, when benzoic acid was used as the substrate (A) and water was used as a quencher, 2, 5-cyclohexadiene-1-carboxylic acid was preferentially produced as described in Example B1 given later. When methyl iodide was used as a quencher and mechanochemical treatment was further performed, 1-methyl-2.5-cyclohexadiene-1-carboxylic acid was preferentially produced as described in Example B51 given later.

### <Aromatic Heterocyclic Compound>

In the reduction method according to the present invention, the aromatic heterocyclic compound used as the substrate (A) is not particularly limited as long as it has, in its molecule, at least one aromatic heterocycle having one or more heteroatoms as ring-constituting atoms. In the aromatic heterocyclic compound, the number of atoms forming the aromatic heterocycle is 4 or more and 30 or less, preferably 6 or more and 14 or less. The number and position of heteroatoms constituting the aromatic heterocycle are not limited. The heteroatom includes an oxygen atom, a nitrogen atom, and a sulfur atom. The aromatic heterocycle may be an aromatic heterocyclic compound having 1 or more and 29 or less carbon atoms and 1 or more and 29 or less heteroatoms. When a plurality of heteroatoms are included, the heteroatoms may be the same atoms or different atoms. The aromatic heterocycle may constitute a fused ring or a spiro ring.

The aromatic heterocycle may be, for example, at least one selected from the group consisting of nitrogen-containing aromatic heterocycles such as pyrroline, pyrazole, pyridine, imidazole, pyrazine, pyrimidine, pyridazine, indolizinyl, isoindole, indole, indazole, purine, isoquinoline, quinoline, phthalazine, naphthyridine, quinoxaline, quinazoline, cinnoline, carbazole, carboline, phenathridine, acridine, perimidine, phenanthroline, and phenazine, oxygen-containing aromatic heterocycles such as furan, benzofuran, isobenzofuran, and benzopyran, sulfur-containing aromatic heterocycles such as thiophene, benzothiophene, and thianthrene, nitrogen- and oxygen-containing aromatic heterocycles such as oxazoline, isoxazoline, furazan, benzoxazole, and benzoisoxazole, nitrogen- and sulfur-containing aromatic heterocycles such as thiazole, isothiazole, benzothiazole, benzoisothiazole, and phenothiazine, and oxygen- and sulfur-containing aromatic heterocycles such as phenoxathiine.

In the reduction method according to the present invention, when acridine is used as the substrate (A), 9,10-dihydroacridine can be produced as described in Example B48 given later.

In the reduction method according to the present invention, when N-methylindole is used as the substrate (A), 1-methyl-2,3-dihydroindole can be produced as described in Example B49 given later.

In the reduction method according to the present invention, when indole is used as the substrate (A), 4, 7-dihydro-1H-indole can be produced as described in Example B50 given later.

### <Internal Alkyne-Containing Compound>

In the reduction method according to the present invention, the internal alkyne compound used as the substrate (A) is not particularly limited as long as it is a compound represented by the following structure (1);

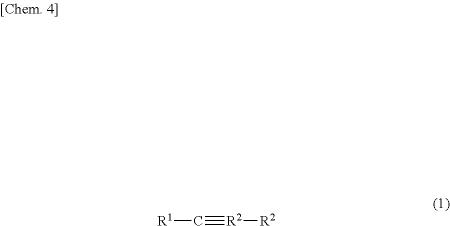

(in the formula, R¹ and R² are monovalent organic groups that may be the same as or different from each other).

In the reduction method according to the present invention, the internal alkyne compound reacts as shown below.

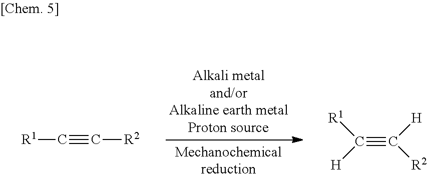

(In formula (1), R¹ and R² are monovalent organic groups that may be the same as or different from each other)

The reduction method according to the present invention can selectively produce an E-olefin compound (trans-alkene compound) from the internal alkyne compound.

### <Conjugated Alkene Compound>

In the reduction method according to the present invention, the conjugated alkene compound used as the substrate (A) is not particularly limited as long as it is, for example, a compound having, in its molecule, a structure represented as any of the following structures (2) to (4);

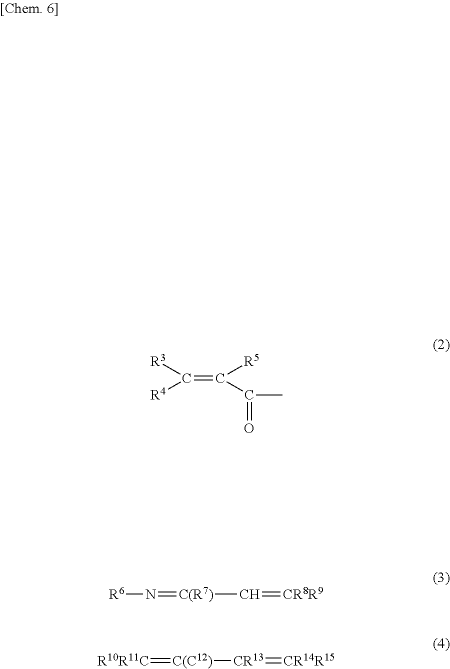

(in formulas (2) to (4), R³ to R¹⁵ are monovalent organic groups that may be the same as or different from each other.).

Of these, the conjugated alkene compound having, in its molecule, a structure represented by formula (2) may be, for example, an α, β-unsaturated carbonyl compound that is at least one selected from the group consisting of α, β-unsaturated carboxylic acids such as acrylic acid, methacrylic acid, and itaconic acid; (meth)acrylic acid ester compounds such as methyl acrylate, methyl methacrylate, ethylene glycol dimethacrylate, and ethylene glycol dimethacrylate; (meth)acrylamide compounds such as acrylamide and methacrylamide; and the like.

The conjugated alkene compound having a structure represented by formula (3) or formula (4) may be, for example, at least one selected from the group consisting of N-cinnamylideneaniline, 1,3-butadiene, isoprene, 1,3-pentadiene, 2,3-dimethylbutadiene, 2, 4-hexadiene, 1,4-diphenylbutadiene, 1,3-heptadiene, 2, 5-dimethyl-2, 4-hexadiene, chloroprene, 2, 4-heptadiene, 2, 4-octadiene, and the like.

In the reduction method according to the present invention, the reduction of the α, β-unsaturated carbonyl compound among the conjugated alkene compounds is, for example, as shown below.

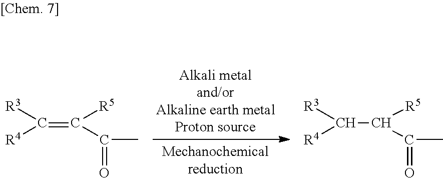

(In the formula, R³ to R⁵ are monovalent organic groups that may be the same as or different from each other.)

### [Metal (B)]

The metal (B) used in the reduction method according to the present invention includes an alkali metal and/or an alkaline-earth metal.

The alkali metal may be at least one selected from the group consisting of lithium, sodium, potassium, rubidium, and cesium. Of these, at least one selected from the group consisting of lithium, sodium, and potassium is preferred, lithium and/or sodium is more preferred, and sodium is still more preferred.

The alkaline-earth metal may be at least one selected from the group consisting of magnesium, calcium, strontium, and barium. Of these, at least one selected from the group consisting of magnesium, calcium, and strontium is preferred, and magnesium and/or calcium is more preferred.

The shape and the like of the metal (B) are not particularly limited. Those skilled in the art can appropriately select and determine them according to reaction conditions such as a scale and a vessel. For example, a metal piece (metal lump) obtained by processing bulk metal or wire metal to be within (0.1 mm to 10 mm) long×(0.1 mm to 10 mm) wide×(0.1 mm to 10 mm) thick can be used.

In the reduction method according to the present invention, the amount of the metal (B) used is not particularly limited. Relative to the substrate, it is, for example, 1.0 equiv (1.0 equivalent) or more, preferably 1.5 equiv or more, more preferably 2.0 equiv or more, and is, for example, 10.0 equiv or less, preferably 8.0 equiv or less.

### [Proton Source (C)]

The proton source (C) used in the reduction method according to the present invention is not particularly limited as long as it is a compound that can release a hydrogen atom in the form of a proton.

The proton source may be, for example, a compound having at least one group that can release a hydrogen atom in the form of a proton. The group that can release a hydrogen atom in the form of a proton may be, for example, a hydroxyl group, an amino group, or a carboxyl group.

The proton source may be, for example, at least one selected from the group consisting of an alcohol compound, an amine compound, an amino alcohol compound, a carboxylic acid compound, an aminocarboxylic acid compound, an ammonium salt, and the like.

The alcohol compound is a compound having one or more hydroxyl groups, and may be, for example, at least one selected from the group consisting of monofunctional alcohols such as methanol, ethanol, n-propanol, i-propanol, n-butanol, tert-butanol, 2-methyl-2-propanol, 1-adamantanol, 2-adamantanol, and cyclohexanol; polyfunctional alcohols or sugar compounds such as ethylene glycol, 1,2-propanediol, 1,3-propanediol, 1,2-butanediol, 1, 3-butanediol, 1,4-butanediol, 1,2-pentanediol, 1,3-pentanediol, 1,5-pentanediol, 1,6-hexanediol, 1,7-heptanediol, 1,8-octanediol, 1,9-nonanediol, 1, 10-decanediol, 1, 12-dodecanediol, 2-methyl-1,3-propanediol, neopentyl glycol, 3-methyl-1,5-pentanediol, 2-methyl-1, 3-pentanediol, 1,3-cyclohexanediol, 1,4-cyclohexanediol, 1,4-cyclohexanedimethanol, 1,2-cyclopentanediol, 1,3-cyclopentanediol, 1,1-cyclohexanedimethanol, 1,2-cyclohexanedimethanol, 1,3-cyclohexanedimethanol, 1,4-cyclohexanedimethanol, 1,2-cyclooctanediol, 1,5-cyclooctanediol, 5-norbornene-2,2-dimethanol, 5-norbornene-2, 3-dimethanol, norbornane-2,3-dimethanol, norbornane-2, 5-dimethanol, 2, 6-decahydronaphthalenedimethanol, 1,3-adamantanediol, 1,4-adamantanediol, 2, 4-adamantanediol, tricyclodecanedimethanol, glycerol, trimethylolpropane, trimethylolethane, hexanetriol, pentaerythritol, dipentaerythritol, 1, 3, 5-cyclohexanetriol, sorbitol, mannitol, sorbitan, diglycerol, triglycerol, tetraglycerol, polypentaglycerol, sucrose, glucose, mannose, fructose, cellulose, and methyl glucoside; and the like.

The amine compound is a compound having one or more amino groups, and may be, for example, at least one selected from the group consisting of aliphatic amines such as methylamine, ethylamine, n-propylamine, isopropylamine, diisopropylamine, triisopropylamine, n-butylamine, tert-butylamine, sec-butylamine, 2-ethylhexylamine, n-octylamine, N-methylethylamine, dimethylamine, diethylamine, triethylamine, ethylenediamine, and propylenediamine; aromatic amines such as aniline, o-tolidine, 2, 4, 6-trimethylaniline, anisidine, 3-(trifluoromethyl) aniline, and phenylenediamine;

heterocyclic amines such as piperazine, pyridine, piperidine, triazine, and melamine; and

the like.

The amino alcohol compound is a compound having one or more amino groups and one or more hydroxyl groups, and may be, for example, at least one selected from the group consisting of ethanolamine, propanolamine, butanolamine, dimethanolamine, trimethanolamine, diethanolamine, triethanolamine, N-methyldiethanolamine, dipropanolamine, tripropanolamine, diisopropanolamine, triisopropanolamine, trishydroxymethylaminomethane, 2-amino-2-methyl-1, 3-propanediol, and the like.

The carboxylic acid compound is an aliphatic compound having one or more carboxyl groups, and may be, for example, at least one selected from the group consisting of formic acid, acetic acid, propionic acid, pivalic acid, trifluoroacetic acid, cyclohexanecarboxylic acid, and the like.

The aminocarboxylic acid compound is a compound having one or more amino groups and one or more carboxyl groups, and may be, for example, at least one selected from the group consisting of glycine, alanine, valine, leucine, isoleucine, serine, threonine, aspartic acid, glutamic acid, asparagine, glutamine, cysteine, cystine, methionine, lysine, hydroxylysine, histidine, arginine, and the like.

In the reduction method according to the present invention, it is preferable to use a proton source (C) having a melting point of 30° C. or higher, preferably 50° C. or higher. In this case, when a reduction reaction is carried out at normal temperature (25° C.), the proton source is present as a solid in the reaction system, and can function not only as the proton source but also as a grinding accelerator for the substrate (A) and/or the metal (B).

In the reduction method according to the present invention, it is preferable to use an alcohol compound or an amine compound as the proton source (C). When sodium is used as the metal (B), it is particularly preferable to use an alcohol compound, particularly, a sugar compound. When lithium is used as the metal (B), it is preferable to use an amine compound and/or an alcohol compound.

In the reduction method according to the present invention, the amount of the proton source (C) used is not particularly limited. Relative to the substrate, it is, for example, 1.0 equiv (1.0 equivalent) or more, preferably 1.5 equiv or more, more preferably 2.0 equiv or more, and is, for example, 20.0 equiv or less, preferably 15.0 equiv or less, more preferably 12.0 equiv or less.

[Other components (D)]

In the reduction method according to the present invention, “other components (D)” other than the substrate (A), the metal (B), and the proton source (C) can be used. As the other components (D), for example, components usable as one or more selected from the group consisting of an ion trapping agent, a reaction terminator (quencher), and a grinding accelerator can be used.

The ion trapping agent is not particularly limited as long as it can trap and stabilize metal ions produced through the use of the metal (B), particularly, alkali metals, calcium, strontium, and barium, in a reduction reaction. For example, one or more solvents having a melting point of lower than 30° C., preferably 20° C. or lower, can be used.

The metal stabilizer may be an organic solvent that is used when Birch reduction is carried out in a solution system. It may be, for example, at least one selected from the group consisting of oxygen-containing organic solvents such as diethyl ether, diisopropyl ether, dibutyl ether, t-butylmethyl ether, tetrahydrofuran, tetrahydropyran, cyclopentylmethyl ether, dimethoxyethane, 1,4-dioxane, anisole, acetoxy-2-ethoxyethane, propylene glycol monomethyl ether acetate, ethylene glycol dimethyl ether, diethylene glycol dimethyl ether, 1-methoxy-1, 1, 2, 2-tetrafluoroethane, 1-ethoxy-1, 1, 2, 2-tetrafluoroethane, acetone, methyl ethyl ketone, ethyl acetate, butyl acetate, and 15-crown 5-ether; aromatic solvents such as benzene, toluene, xylene, mesitylene durene, and decalin; aliphatic organic solvents such as hexane, pentane, and heptane; halogenated hydrocarbon organic solvents such as dichloromethane, chloroform, carbon tetrachloride, chlorobenzene, and 1, 2-dichlorobenzene; nitrogen-containing organic solvents such as 1, 3-dimethyl-2-imidazolidinone, acetonitrile, N, N-dimethylformamide, N, N-dimethylacetamide, N-methyl-2-pyrrolidone, and pyridine; sulfur-containing organic solvents such as dimethyl sulfoxide, phosphorus-containing organic solvents such as hexamethylphosphoric triamide, and the like.

The reaction terminator (quencher) is not particularly limited as long as it can terminate a reduction reaction. Examples include those containing at least one selected from the group consisting of water, methyl iodide, methylene chloride, acetone, methyl ethyl ketone, ethyl acetate, butyl acetate, and the like. In the present invention, it is preferable to use a reaction terminator (quencher) containing water.

The grinding accelerator (grinding aid) is not particularly limited as long as it can perform, in mechanochemical treatment, grinding acceleration or reaction acceleration by the mechanochemical treatment on at least any one or more of the substrate (A), the metal (B), and the proton source (C).

The grinding accelerator may be used as the proton source (C) and/or the metal stabilizer.

As the grinding accelerator, for example, at least one compound (compound that is solid at normal temperature (25° C.)) having a melting point of 30° C. or higher, preferably 50° C. or higher, can be used. It may be, for example, at least one selected from the group consisting of inorganic particles such as silica, alumina, zirconia, calcium carbonate, ceramic, glass beads, and sea sand, and organic particles such as sucrose, glucose, mannose, fructose, and cellulose. The average particle size of these grinding accelerators is typically 1 μm or more, preferably 10 μm or more, and is, for example, 1 mm or less. When a compound that is solid at normal temperature (25° C.) is used as the grinding accelerator, the reaction can be efficiently carried out even when compounds that are liquid at normal temperature are used as the substrate (A) and/or the proton source (C).

As the grinding accelerator (grinding aid), for example, at least one solvent having a melting point of lower than 30° C., preferably 20° C. or lower, can be used as liquid assisted grinding (LAG). The LAG may be, for example, at least one selected from the group consisting of the solvents exemplified as being usable as the metal stabilizer.

In the present invention, when an organic solvent having a melting point of lower than 30° C. is contained as the proton source (C) and/or the other component (D) in the reaction system, it is preferred that the amount of the organic solvent used be 1.0 mL or less, preferably 0.8 mL or less, based on the total amount of the substrate taken as 1 mmol. The lower limit of the amount of the organic solvent used may be 0 mL (no organic solvent used). The amount of the organic solvent used in the mechanochemical treatment corresponds to any of the cases where the organic solvent is not used at all, the solvent is not actively used, and the organic solvent is used but in such a very small amount that the solvent effect is not exhibited.

In these cases, the substrate (A), the metal (B), the proton source (D), and the other components (D) are present, at the time of the mechanochemical treatment (the start of the reaction), typically in a state of being at least partially not dissolved in the organic solvent or the like, occasionally in a solid state of being not dissolved in the organic solvent or the like at all, and react with each other. In general, when a reaction is carried out in a solution system, an organic solvent in an amount of more than 1 mL, preferably 10 mL or more, relative to 1 mmol of the total amount of a substrate is used. In the present invention, the amount of the organic solvent used is 1.0 mL or less relative to 1 mmol of the total amount of the substrate.

### [Mechanochemical Treatment]

In the reduction method according to the present invention, the mechanochemical treatment is a treatment for directly applying mechanical energy mechanically generated by means of grinding, shearing, impact, compression, or the like to at least the substrate (A), the metal (B), and the proton source (C) to thereby reduce the substrate. The mechanochemical treatment can be carried out under solvent-free or substantially solvent-free conditions, contributes to waste reduction and has a low environmental load, and also does not require complicated reaction condition setting or preparation. Furthermore, the reduction reaction can be carried out simply and in a very short time, thus enabling achievement of both suppression of energy consumption and high productivity.

The reaction device and the like used in the mechanochemical treatment and various conditions and the like in the mechanochemical treatment can be as follows.

### <Reaction Device>

The reaction device used in the mechanochemical treatment is not particularly limited as long as it is a device which can apply mechanical energy to at least the substrate (A), the metal (B), and the proton source (C) and with which the reduction reaction of the substrate (A) can be carried out.

Examples of such devices include one or more selected from the group consisting of:
mills such as ball mills, rod mills, jet mills, and SAG mills;
grinders such as rotary millstones and grinding machines;
(horizontal-axis rotating) rotary vessel type mixing devices of horizontal cylinder type, V type, double cone type, cube type, S type, continuous V type, and other types;
rotary vessel type mixing devices (with baffle plates and blades) of horizontal cylinder type, V type, double cone type, ball mill type, and other types;
(rotationally oscillating) rotary vessel type mixing devices of rocking type, cross-rotary type, and other types;
(horizontal-axis rotating) fixed vessel type mixing devices of ribbon type, paddle type, single-axis rotor type, bug mill type, and other types;
(vertical-axis rotating) fixed vessel type mixing devices of ribbon type, screw type, planetary type, turbine type, high-speed flow type, rotating disk type, muller type, and other types;
(vibratory) fixed vessel type mixing devices of vibratory mill type, sieve type, and other types;
(fluidal) hydrokinetic mixing devices of heterogeneous fluidized bed type, swirling fluidized bed type, riser tube type, jet pump type, and other types;
(gravitational) hydrokinetic mixing devices of gravity type, static mixer type, and other types;
kneaders such as twin-screw kneaders, single-screw kneaders, mixers, and roll mills; and the like.

As the device used in the mechanochemical treatment, one or more selected from the group consisting of mills, grinders, mixing devices, kneaders, and the like are preferably used, and a mixing device is particularly preferably used. For the mixing device, for example, reference can be made to the powder mixing devices described in Table 5,

FIG. 9, etc. of Sakashita “Mixture Process Technology for the Powders” Journal of the Japan Society of Colour Material, 77 (2), 75-85 (2004). Specifically, a ball mill, a twin-screw kneader, a planetary ball mill, a SPEX mixer mill, a twin-screw ball mill, or the like can be used.

The device used in the mechanochemical treatment may include one or more means selected from the group consisting of measuring means, depressurizing or pressurizing means, atmosphere adjusting means (gas introduction or discharge means), means for introducing various components, means for discharging various components and reaction products, purification means, analysis means, reaction monitoring means, and the like.

### <Reaction Vessel>

The reaction vessel used in the mechanochemical treatment is not particularly limited. An appropriate reaction vessel is selected and used in consideration of the physical properties, reactivity, and amount of the substrate (A), the metal (B), the proton source (C), and other components present in the reaction system, the reaction conditions in the mechanochemical treatment, etc. For example, when a device that performs mixing treatment in a mechanical manner (e.g., a ball mill) is used, a ball mill jar or the like can be used as the reaction vessel. The inner capacity of the reaction vessel such as a ball mill jar is not particularly limited. For example, the inner capacity can be 1 mL or more, and, for example, the inner capacity can be 10 mL.

The reaction vessel used in the mechanochemical treatment may include a stirring means for stirring the substrate (A), the metal (B), the proton source (C), and other components present in the reaction system. The stirring means that the reaction vessel may include is not particularly limited as long as it is a stirring means that can be provided in the reaction device. A means using the device that performs mixing treatment in a mechanical manner described in (Reaction device) above can be used. As the device that performs mixing treatment in a mechanical manner, for example, a ball mill is preferably used.

### <Conditions of Mechanochemical Treatment>

The conditions of the mechanochemical treatment are not particularly limited, and appropriate conditions are employed in consideration of the physical properties, reactivity, and amount of the substrate (A), the metal (B), the proton source (C), and other components present in the reaction system, the reaction conditions in the mechanochemical treatment, etc.

By mechanochemically treating at least the substrate (A), the metal (B), and the proton source (C) under appropriate conditions, the substrate (A), the metal (B), and the proton source (C) are activated by the mechanical action of the mechanochemical treatment, and the reduction reaction of the substrate (A) is carried out.

### (Mechanical Energy Applied)

In the mechanochemical treatment, the amount of mechanical energy applied is not particularly limited as long as it is a mechanical energy amount at which the reduction reaction of the substrate (A) can be carried out by reacting at least the substrate (A), the metal (B), and the proton source (C).

For example, when the device used in the mechanochemical treatment is a mixing device, the mixing rate is not particularly limited. The mixing rate can be appropriately determined in consideration of the physical properties, reactivity, and amount of the substrate (A), the metal (B), the proton source (C), and other components present in the reaction system, the reaction conditions in the mechanochemical treatment, etc. For example, when a ball mill is used, the vibration frequency in shaking and stirring is, for example, 5 Hz or more, preferably 10 Hz or more, more preferably 20 Hz or more, and can be, for example, 100 Hz or less, preferably 80 Hz or less.

### (Temperature)

In the mechanochemical treatment, the temperature in the reaction vessel (the temperature in the reaction system) is not particularly limited as long as it is a temperature at which at least the reduction reaction of the substrate (A) can be carried out. The temperature can be appropriately determined in consideration of the physical properties, reactivity, and amount of the substrate (A), the metal (B), the proton source (C), and other components present in the reaction system, the reaction conditions in the mechanochemical treatment, etc. For example, the temperature is 0° C. or higher, preferably 10° C. or higher, and is, for example, 500° C. or lower, preferably 300° C. or lower, more preferably 250° C. or lower. In the present invention, the mechanochemical treatment can be carried out at room temperature (25° C.).

The method of controlling the temperature is not particularly limited. Various temperature control methods used in conducting chemical reactions can be used. Examples include controlling the temperature in the reaction vessel (reaction system) by heating or cooling the reaction vessel itself using a heating or cooling means, controlling the temperature in the reaction vessel by covering the reaction vessel with a heating medium at a predetermined temperature, and controlling the temperature in the reaction vessel by providing a heating element or a cooling element. When the reaction is carried out under heating conditions, controlling the temperature in the reaction vessel by applying warm air generated with a heat gun to the reaction vessel may be used, for example, from the viewpoint of safety and ease of temperature control operation.

### (Pressure)

In the mechanochemical treatment, the pressure in the reaction vessel (the pressure in the reaction system) is not particularly limited as long as it is a pressure at which at least the reduction reaction of the substrate (A) can be carried out. The pressure can be appropriately determined in consideration of the physical properties, reactivity, and amount of the substrate (A), the metal (B), the proton source (C), and other components present in the reaction system, the reaction conditions in the mechanochemical treatment, etc. For example, any of a substantially vacuum state, a reduced-pressure state, a pressurized state, and an atmospheric-pressure state may be employed. If the mechanochemical treatment can be carried out in the atmospheric-pressure state without pressurization and depressurization, the atmospheric-pressure state is preferred, for example, from the viewpoint of operability, reaction device, cost, safety, etc.

The method of controlling the pressure is not particularly limited. When the reaction vessel (reaction system) is pressurized or depressurized, various pressure control methods used in conducting chemical reactions can be used. For example, the pressure in the reaction vessel (the pressure in the reaction system) can be increased or reduced using a pressurizing or depressurizing means.

### (Atmosphere)

In the mechanochemical treatment, the atmosphere in the reaction vessel (the atmosphere in the reaction system) is not particularly limited as long as it is an atmosphere in which at least the reduction reaction of the substrate (A) can be carried out. The atmosphere can be appropriately determined in consideration of the physical properties, reactivity, and amount of the substrate (A), the metal (B), the proton source (C), and other components present in the reaction system, the reaction conditions in the mechanochemical treatment, etc. For example, the mechanochemical treatment can be carried out in an air atmosphere, which is a non-inert atmosphere, without any particular atmosphere adjustment. If necessary, the mechanochemical treatment can be carried out in an inert gas atmosphere such as nitrogen, helium, neon, or argon. In the present invention, the mechanochemical treatment can be carried out typically in an air atmosphere, which is a non-inert atmosphere.

### (Treatment)

In the mechanochemical treatment, the treatment time is not particularly limited as long as it is a treatment time during which at least the reduction reaction of the substrate (A) can be carried out. The treatment time can be appropriately determined in consideration of the physical properties, reactivity, and amount of the substrate (A), the metal (B), the proton source (C), and other components present in the reaction system, the reaction conditions in the mechanochemical treatment, etc. The treatment time can be, for example, 15 seconds or more, typically 30 seconds or more, preferably 1 minute or more, more preferably 3 minutes or more, still more preferably 5 minutes or more. The upper limit of the reaction time is not particularly limited, and can be, for example, 24 hours or less, preferably 12 hours or less, more preferably 6 hours or less, still more preferably 1 hour or less, yet still more preferably 30 minutes or less.

(Input Order of Substances to be Treated and Substrate in Treatment, Treatment after Reaction, Etc.)

In the mechanochemical treatment, the order in which the substrate (A), the metal (B), the proton source (C), and other components present in the reaction system are put in the reaction vessel is not particularly limited. The means of input is also not particularly limited. For example, all the components may be put into the reaction vessel at once, or some of the components may be put into the reaction vessel and reacted, followed by the input of the remaining components into the reaction vessel.

The reaction product obtained after the reduction reaction of the substrate (A) is carried out by the mechanochemical treatment can be purified as necessary. The purification method is not particularly limited, and, for example, a method such as filtration, distillation, recrystallization, column chromatography, or washing with a solvent is used.

### EXAMPLES

The present invention will now be described in detail with reference to specific examples. These specific examples are merely embodiments of the present invention. The present invention is not limited at all by these examples.

“Equiv” denotes “equivalent”.

In Examples and Comparative Examples, when a reaction was carried out using a ball mill, a stainless-steel ball mill jar was charged with reagents, and an MM400 model ball mill manufactured by Verder Scientific Co., Ltd. (formerly Retsch) was used.

### {Mechanochemical Reduction Using Alkali Metal}

### <<Mechanochemical Reduction Using Metallic Sodium>>

Compounds Aa1 to Aa11 including an unsaturated bond and reaction products Ab1 to Ab11 used in Examples A1 to A28 and Reference Examples A1 to A4 are as shown below. In the structural formulas, “Me” represents a methyl group, “iPr” represents an isopropyl group, and “tBu” represents a tertiary butyl group.

### <Aromatic Hydrocarbon Compound>

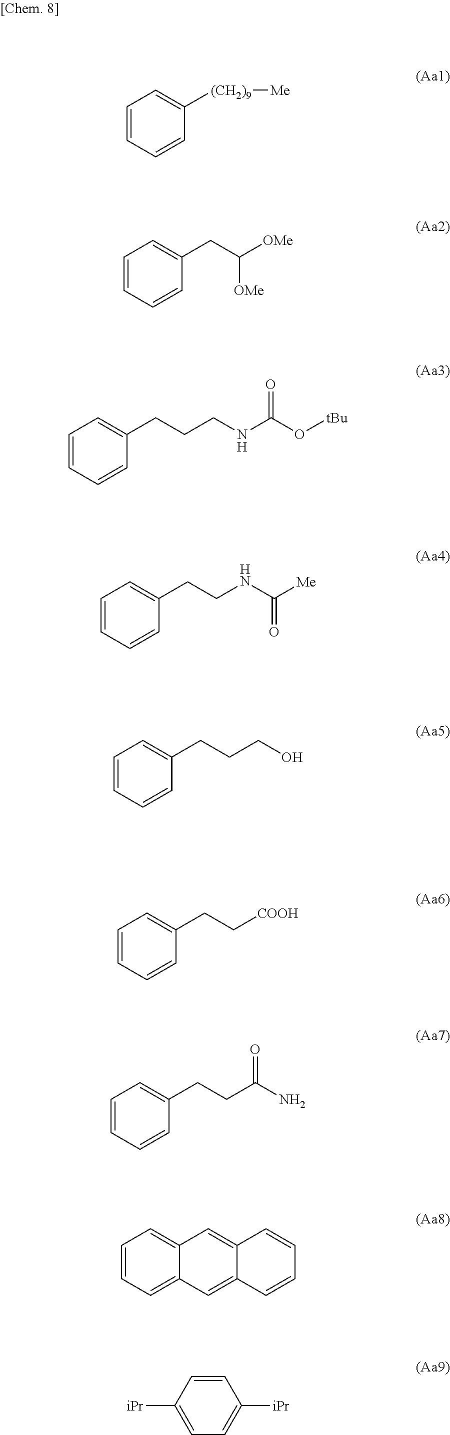

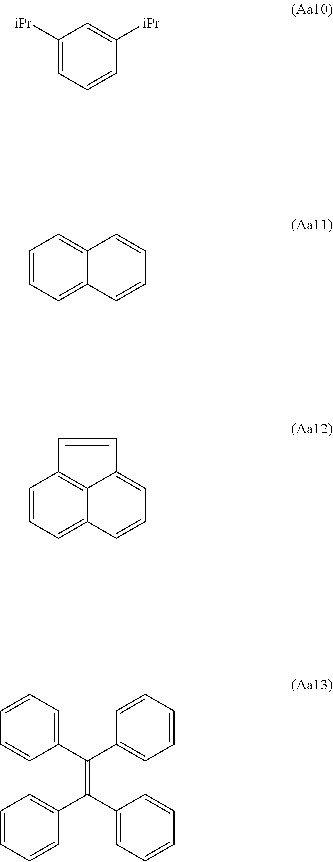

### <Reaction Product>

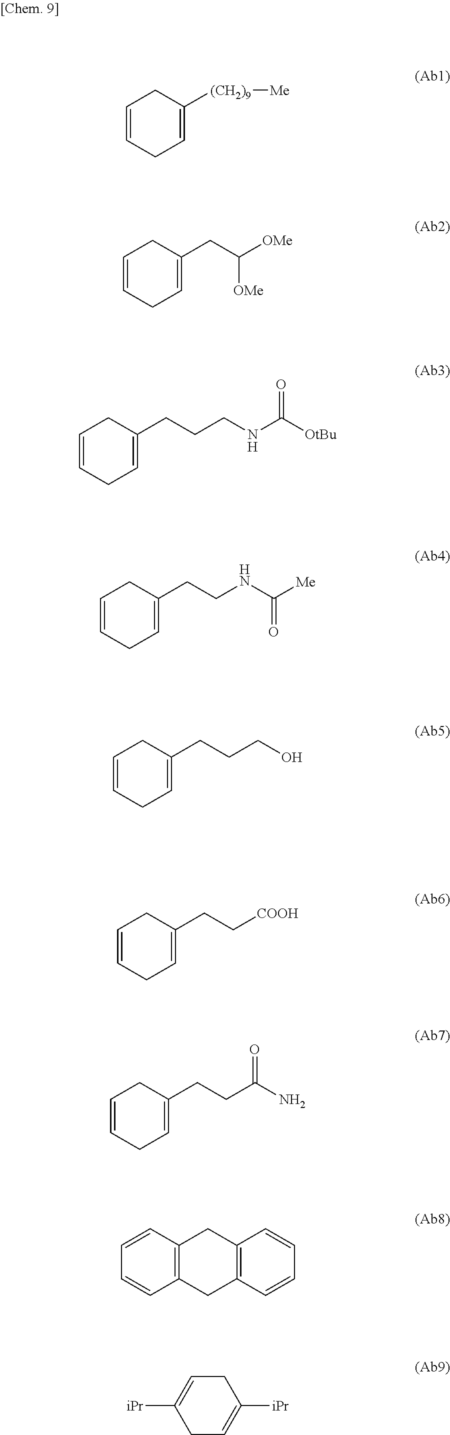

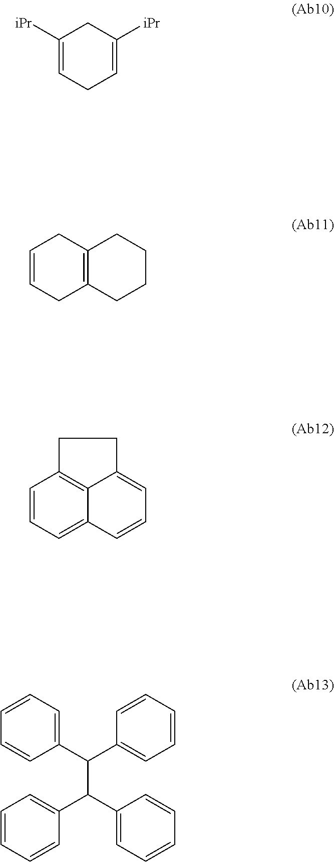

### <Mechanochemical Reduction of Decylbenzene>

### Example A1

A 10 mL stainless-steel ball mill jar containing two stainless-steel balls having a diameter of 10 mm was charged under air with 1.0 mmol of decylbenzene (Aa1) as a substrate, a metallic sodium lump about 3 to 5 mm long, 3 to 5 mm wide, and 3 mm thick in an amount of 6.0 equiv relative to the substrate, 1,3-dimethyl-2-imidazolidinone in an amount of 6.0 equiv relative to the substrate, and D-(+)-glucose as a proton source in an amount of 3.0 equiv relative to the substrate, and the ball mill jar was capped. Thereafter, the ball mill jar was mounted to a ball mill, and a reaction was carried out at room temperature (internal temperature: 25° C.) for 15 minutes by performing shaking and stirring at a vibration frequency of 30 Hz. Then, the ball mill jar was uncapped, and water was added to terminate the reaction, thereby obtaining 1-decyl-1, 4-cyclohexadiene (Ab1) as a reaction product. The isolated yield of the reaction product was 99%.

### Example A2

The reaction was carried out in the same manner as in Example A1 except that the metallic sodium lump in an amount of 4.0 equiv relative to the substrate and 1,3-dimethyl-2-imidazolidinone in an amount of 4.0 equiv relative to the substrate were charged, thereby obtaining a reaction product. The ¹H NMR yield of the reaction product was 86%.

### Example A3

The reaction was carried out in the same manner as in Example A1 except that tert-butanol, as a proton source in place of D-(+)-glucose, in an amount of 3.0 equiv relative to the substrate was charged, thereby obtaining a reaction product. The ¹H NMR yield of the reaction product was 6%.

### Example A4

The reaction was carried out in the same manner as in Example A1 except that tert-butanol, as a proton source in place of D-(+)-glucose, in an amount of 3.0 equiv relative to the substrate and 100 mg of sea sand (particle size: 425 μm to 850 μm) were charged, and the time of the reaction by shaking and stirring was 30 minutes, thereby obtaining a reaction product. The ¹H NMR yield of the reaction product was 18%.

### Example A5

The reaction was carried out in the same manner as in Example A1 except that 2-propanol, as a proton source in place of D-(+)-glucose, in an amount of 3.0 equiv relative to the substrate was charged, thereby obtaining a reaction product. The ¹H NMR yield of the reaction product was 13%.

### Example A6

The reaction was carried out in the same manner as in Example A1 except that 1-adamantanol, as a proton source in place of D-(+)-glucose, in an amount of 3.0 equiv relative to the substrate was charged, thereby obtaining a reaction product. The ¹H NMR yield of the reaction product was 18%.

### Example A7

The reaction was carried out in the same manner as in Example A1 except that cellulose, as a proton source in place of D-(+)-glucose, in an amount of 3.0 equiv relative to the substrate was charged, thereby obtaining a reaction product. The ¹H NMR yield of the reaction product was 13%.

### Example A8

The reaction was carried out in the same manner as in Example A1 except that hexamethylphosphoric triamide in an amount of 6.0 equiv relative to the substrate was charged in place of 1, 3-dimethyl-2-imidazolidinone, thereby obtaining a reaction product. The ¹H NMR yield of the reaction product was 19%.

### Example A9

The reaction was carried out in the same manner as in Example A1 except that 15-crown 5-ether in an amount of 6.0 equiv relative to the substrate was charged in place of 1,3-dimethyl-2-imidazolidinone, thereby obtaining a reaction product. The ¹H NMR yield of the reaction product was 23%.

### Example A10

The reaction was carried out in the same manner as in Example A1 except that the number of stainless-steel balls having a diameter of 10 mm was one, thereby obtaining a reaction product. The ¹H NMR yield of the reaction product was 85%.

### Example A11

The reaction was carried out in the same manner as in Example A1 except that the reaction was carried out by performing shaking and stirring at a vibration frequency of 25 Hz, thereby obtaining a reaction product. The ¹H NMR yield of the reaction product was 72%.

### Example A12

The reaction was carried out in the same manner as in Example A1 except that the time of the reaction by shaking and stirring was 5 minutes, thereby obtaining a reaction product. The ¹H NMR yield of the reaction product was 61%.

### Reference Example A1

A reaction vessel was charged under air with 1.0 mmol of decylbenzene (Aa1) as a substrate, metallic sodium in an amount of 6.0 equiv relative to the substrate, 1,3-dimethyl-2-imidazolidinone in an amount of 6.0 equiv relative to the substrate, D-(+)-glucose as a proton source in an amount of 3.0 equiv relative to the substrate, and tetrahydrofuran (THF) in an amount such that the concentration of decylbenzene (Aa1) became 0.28 M (0.28 mol/L), a reaction was carried out by performing stirring at room temperature (25° C.) for 15 minutes, and water was added to the reaction vessel to terminate the reaction. The ¹H NMR yield of 1-decyl-1, 4-cyclohexadiene (Ab1), a reaction product, was less than 5%.

### Reference Example A2

The reaction was carried out in the same manner as in Example A1 except that a metallic sodium dispersion (dispersion solvent: mineral oil) was charged in place of metallic sodium such that the amount of metallic Na was 6.0 equiv relative to the substrate. The ¹H NMR yield of 1-decyl-1, 4-cyclohexadiene (Ab1), a reaction product, was less than 5%.

### Reference Example A3

A reaction vessel was charged under air with 1.0 mmol of decylbenzene (Aa1) as a substrate, metallic sodium in an amount of 4.0 equiv relative to the substrate, 2.6 mL of 1, 3-dimethyl-2-imidazolidinone, tert-butanol as a proton source in an amount of 4.0 equiv relative to the substrate, and 1.0 mL of tetrahydrofuran (THF), a reaction was carried out by performing stirring at room temperature (25° C.) for 15 minutes, and water was added to the reaction vessel to terminate the reaction. The ¹H NMR yield of 1-decyl-1, 4-cyclohexadiene (Ab1), a reaction product, was less than 5%.

### Reference Example A4

A reaction vessel was charged under air with 1.0 mmol of decylbenzene (Aa1) as a substrate, metallic sodium in an amount of 9.0 equiv relative to the substrate, 15-crown 5-ether in an amount of 9.0 equiv relative to the substrate, 2-propanol as a proton source in an amount of 9.0 equiv relative to the substrate, and 6.0 mL of tetrahydrofuran (THF), a reaction was carried out by performing stirring at room temperature (25° C.) for 35 minutes, and water was added to the reaction vessel to terminate the reaction. The ¹H NMR yield of 1-decyl-1, 4-cyclohexadiene (Ab1), a reaction product, was less than 5%.

### <Mechanochemical Reduction of Various Substrates>

### Examples A13 to A21

A 10 mL stainless-steel ball mill jar containing two stainless-steel balls having a diameter of 10 mm was charged under air with 1.0 mmol of substrate Aan, a metallic sodium lump in an amount of XA equiv relative to the substrate, 1, 3-dimethyl-2-imidazolidinone in an amount of YA equiv relative to the substrate, and D-(+)-glucose as a proton source in an amount of ZA equiv relative to the substrate, and the ball mill jar was capped. Thereafter, the ball mill jar was mounted to a ball mill, and a reaction was carried out at room temperature (internal temperature: 25° C.) for TA minutes by performing shaking and stirring at a vibration frequency of 30 Hz. Then, the ball mill jar was uncapped, and water was added to terminate the reaction, thereby obtaining reaction product Abn. The kinds of Aan, XA, YA, ZA, TA, and Abn and the yield (isolated yield or ¹H NMR yield) of the reaction products are shown in Table 1.

TABLE 1

| | | | | | | Reaction | |
|---|---|---|---|---|---|---|---|
| | Substrate | X_{A} | Y_{A} | Z_{A} | TA | product | |
| Example | Aan | (equiv) | (equiv) | (equiv) | (min) | Abn | Yield |
| A13 | Aa2 | 6.0 | 6.0 | 3.0 | 15 | Ab2 | 84% (isolated) |
| A14 | Aa3 | 6.0 | 6.0 | 6.0 | 15 | Ab3 | 66% (isolated) |
| A15 | Aa4 | 6.0 | 6.0 | 6.0 | 15 | Ab4 | 84% (isolated) |
| A16 | Aa5 | 6.0 | 6.0 | 6.0 | 5 | Ab5 | 76% (NMR) |
| A17 | Aa6 | 6.0 | 6.0 | 3.0 | 30 | Ab6 | 93% (isolated) |
| A18 | Aa7 | 6.0 | 6.0 | 6.0 | 15 | Ab7 | 51% (NMR) |
| A19 | Aa8 | 2.6 | 6.0 | 3.0 | 15 | Ab8 | 91% (isolated) |
| A20 | Aa9 | 6.0 | 6.0 | 6.0 | 30 | Ab9 | 89% (isolated) |
| A21 | Aa10 | 6.0 | 6.0 | 6.0 | 30 | Ab10 | 44% (NMR) |

### <Mechanochemical Reduction of Naphthalene Using Na>

### Example A22

A 10 mL stainless-steel ball mill jar containing two stainless-steel balls having a diameter of 10 mm was charged under air with 1.0 mmol of substrate Aa11 (naphthalene), a metallic sodium lump in an amount of 6.0 equiv relative to the substrate, 1,3-dimethyl-2-imidazolidinone in an amount of 6.0 equiv relative to the substrate, and D-(+)-glucose as a proton source in an amount of 3.0 equiv relative to the substrate, and the ball mill jar was capped. Thereafter, the ball mill jar was mounted to a ball mill, and a reaction was carried out at room temperature (internal temperature: 25° C.) for 30 minutes by performing shaking and stirring at a vibration frequency of 30 Hz. Then, the ball mill jar was uncapped, and water was added to terminate the reaction, thereby obtaining reaction product Ab11 (1, 2, 3, 4, 5, 8-hexahydronaphthalene). The isolated yield of the reaction product was 57%.

### <Mechanochemical Reduction of Acenaphthylene Using Na>

### Example A23

A 10 mL stainless-steel ball mill jar containing two stainless-steel balls having a diameter of 10 mm was charged under air with 1.0 mmol of substrate Aa12 (acenaphthylene), a metallic sodium lump in an amount of 2.2 equiv relative to the substrate, 1,3-dimethyl-2-imidazolidinone in an amount of 6.0 equiv relative to the substrate, and D-(+)-glucose as a proton source in an amount of 3.0 equiv relative to the substrate, and the ball mill jar was capped. Thereafter, the ball mill jar was mounted to a ball mill, and a reaction was carried out at room temperature (internal temperature: 25° C.) for 15 minutes by performing shaking and stirring at a vibration frequency of 30 Hz. Then, the ball mill jar was uncapped, and water was added to terminate the reaction, thereby obtaining reaction product Ab12 (acenaphthene). The isolated yield of the reaction product was 81%.

### <Mechanochemical Reduction of Tetraphenylethylene Using Na>

### Example A24

A 10 mL stainless-steel ball mill jar containing two stainless-steel balls having a diameter of 10 mm was charged under air with 1.0 mmol of substrate Aa13 (tetraphenylethylene), a metallic sodium lump in an amount of 3.0 equiv relative to the substrate, 1,3-dimethyl-2-imidazolidinone in an amount of 6.0 equiv relative to the substrate, and D-(+)-glucose as a proton source in an amount of 3.0 equiv relative to the substrate, and the ball mill jar was capped. Thereafter, the ball mill jar was mounted to a ball mill, and a reaction was carried out at room temperature (internal temperature: 25° C.) for 15 minutes by performing shaking and stirring at a vibration frequency of 30 Hz. Then, the ball mill jar was uncapped, and water was added to terminate the reaction, thereby obtaining reaction product Ab13 (1, 1, 2, 2-tetraphenylethane). The isolated yield of the reaction product was 78%.

### <Mechanochemical Reduction Using Inorganic Particles>

### Example A25

A 5 mL stainless-steel ball mill jar containing two stainless-steel balls having a diameter of 10 mm was charged under air with 1.0 mmol of decylbenzene (Aa1) as a substrate, a metallic sodium lump in an amount of 6.0 equiv relative to the substrate, 1-adamantanol as a proton source in an amount of 6.0 equiv relative to the substrate, 1,3-dimethyl-2-imidazolidinone in an amount of 6.0 equiv relative to the substrate, and 100 mg of sea sand (particle size: 425 μm to 850 μm), and the ball mill jar was capped. Thereafter, the ball mill jar was mounted to a ball mill, and a reaction was carried out at room temperature (internal temperature: 25° C.) for 30 minutes by performing shaking and stirring at a vibration frequency of 30 Hz. Then, the ball mill jar was uncapped, and water was added to terminate the reaction, thereby obtaining 1-decyl-1, 4-cyclohexadiene (Ab1) as a reaction product. The isolated yield of the reaction product was 93%.

### Examples A26 to A28

Reaction products Ab2, Ab5, and Ab7 were obtained in the same manner as in Example A25 except that Aa2, Aa5, or Aa7 was used in place of Aa1 as a substrate. The yields (isolated yield and/or ¹H NMR yield) of the reaction products are shown in Table 2.

TABLE 2

| | | Reaction | |
|---|---|---|---|
| | Substrate | product | |
| Example | Aan | Abn | Yield |
| A25 | Aa1 | Ab1 | 93% (isolated) |
| A26 | Aa2 | Ab2 | 77% (isolated) |
| | | | 91% (NMR) |
| A27 | Aa5 | Ab5 | 57% (isolated) |
| | | | 91% (NMR) |
| A28 | Aa7 | Ab7 | 62% (NMR) |

### <<Mechanochemical Reduction Using Metallic Lithium>>

Aromatic hydrocarbon compounds Ba1 to Ba38 and reaction products Bb1 to Bb38, Bb1′, and Bb32′ used in Examples B1 to B53 are as shown below. In the structural formulas, “Me” represents a methyl group, “iPr” represents an isopropyl group, “tBu” represents a tertiary butyl group, “TBS” represents a tertiary butyldimethylsilyl group, and “Boc” represents a tertiary butoxycarbonyl group.

The lithium wire used as a metallic lithium source was about 3 mm in length and about 43.2 mm in diameter, and was used after being wiped off adhering oil and cut into small pieces.

### <Aromatic Hydrocarbon Compound>

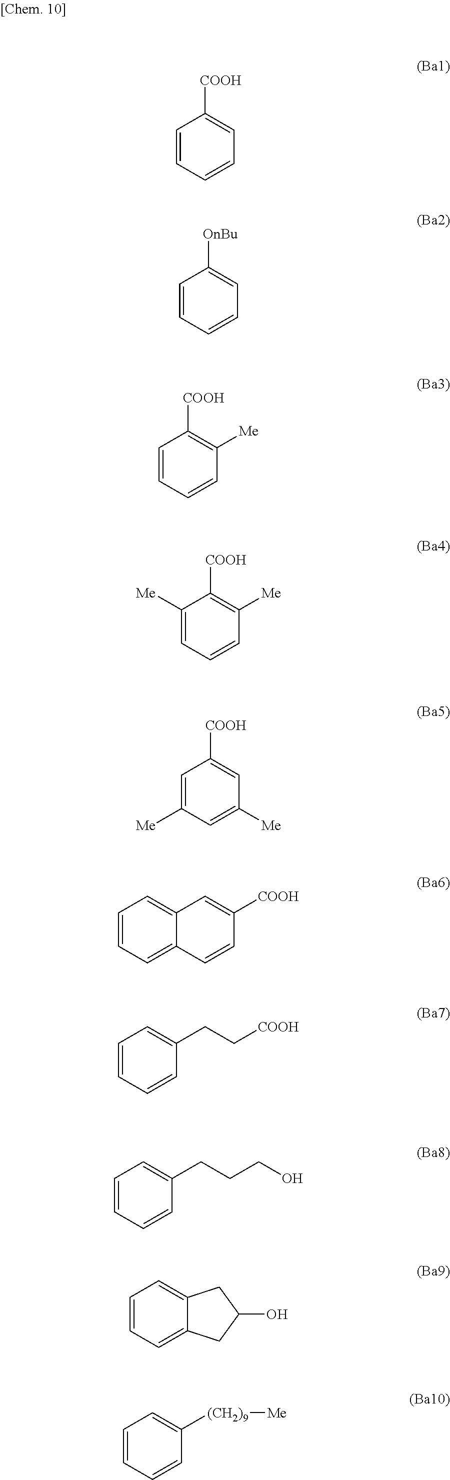

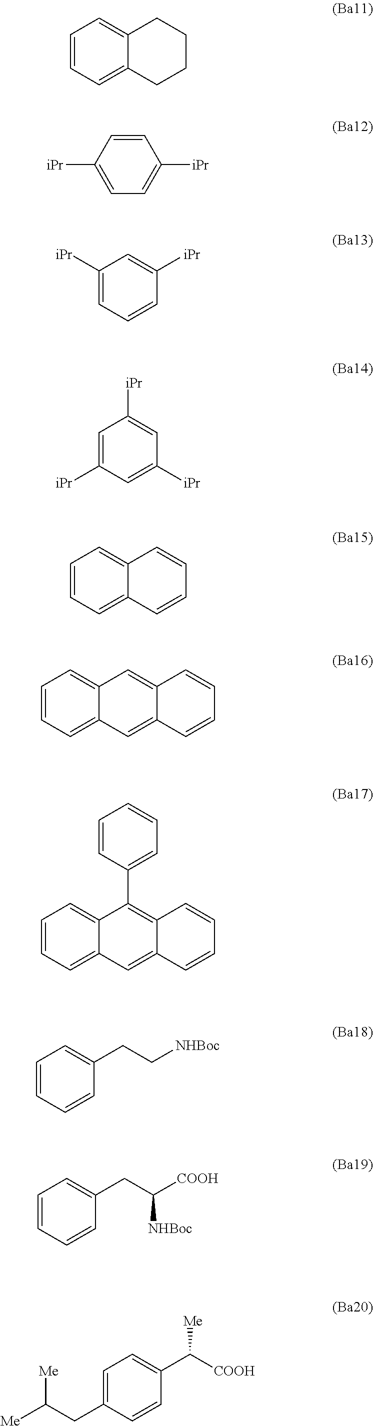

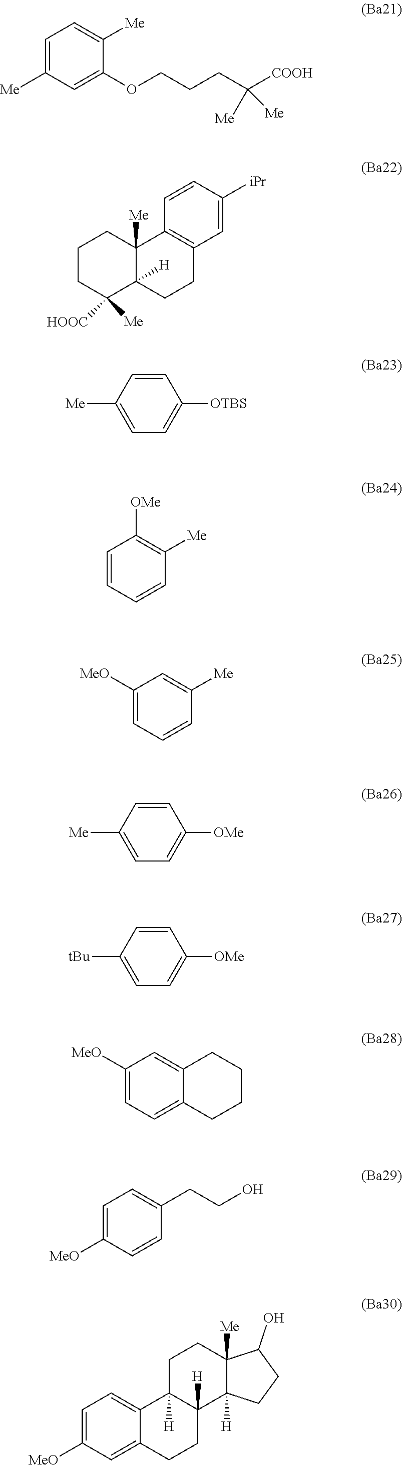

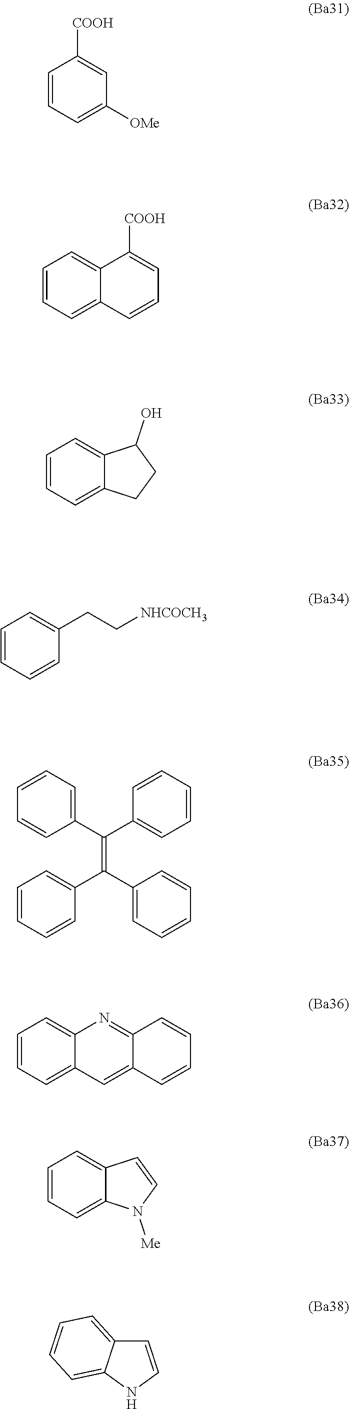

### <Reaction Product>

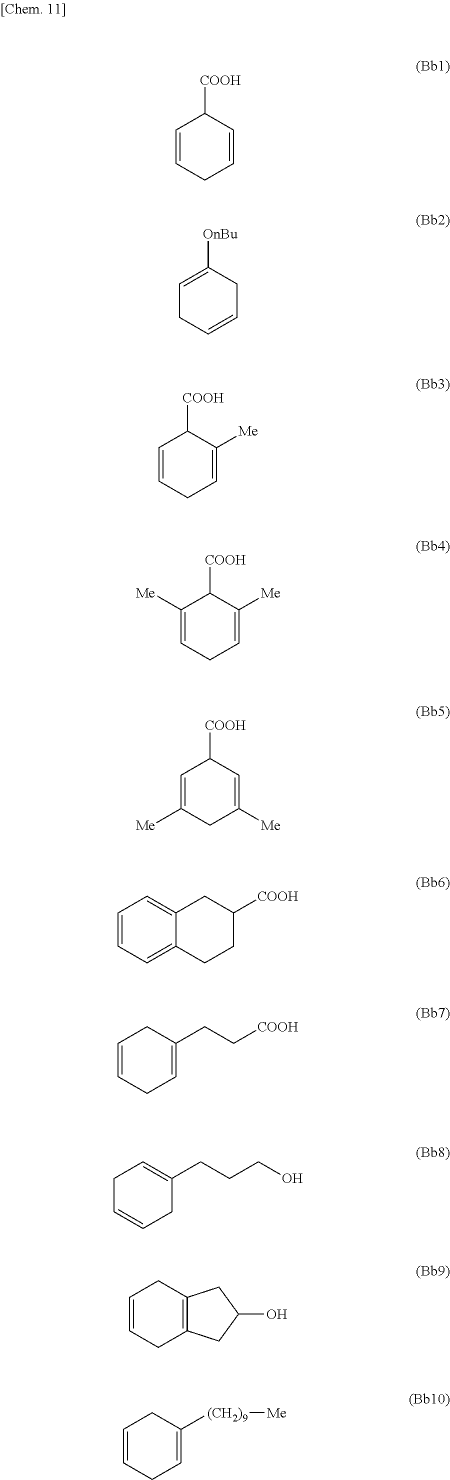

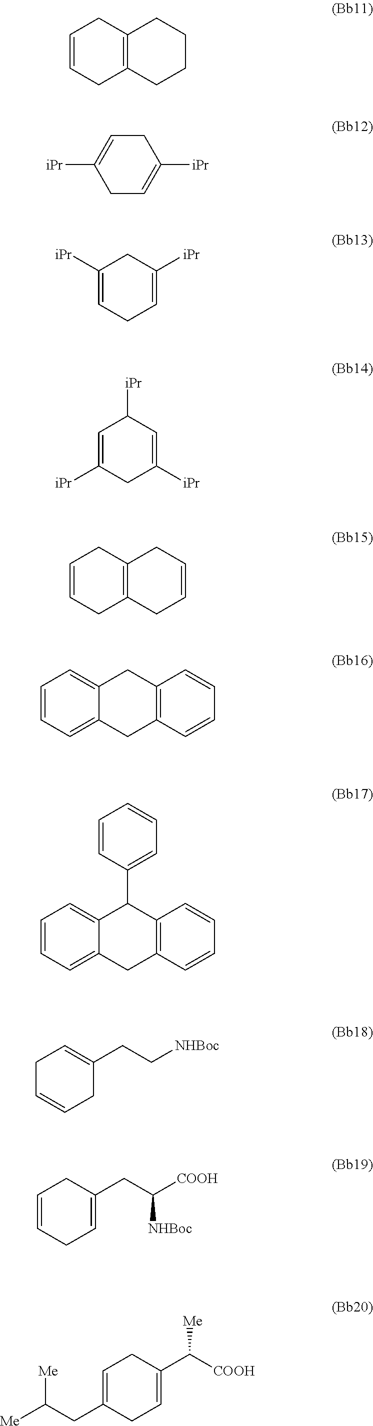

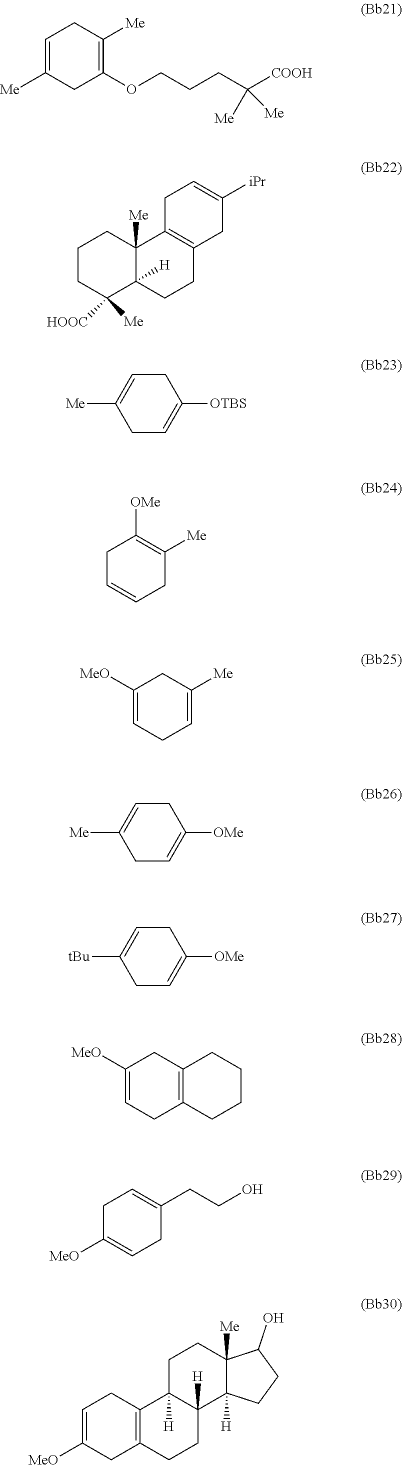

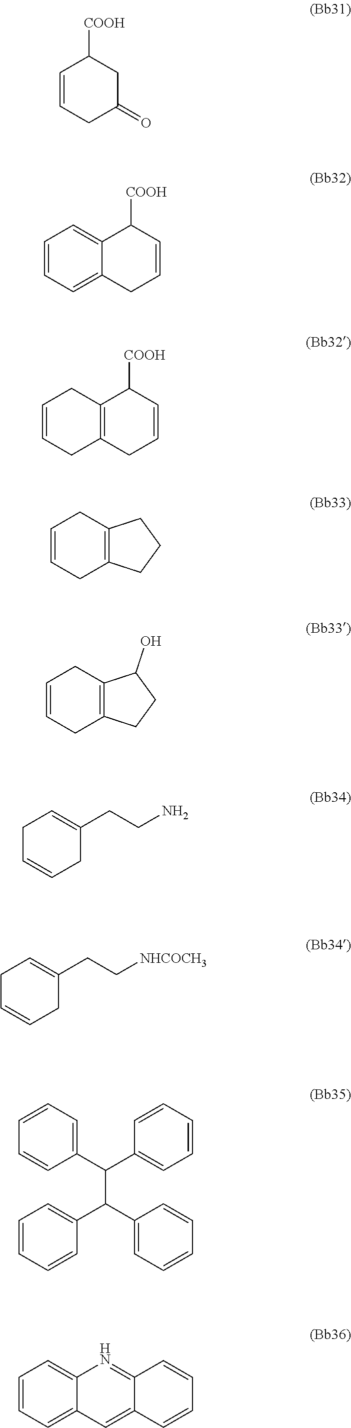

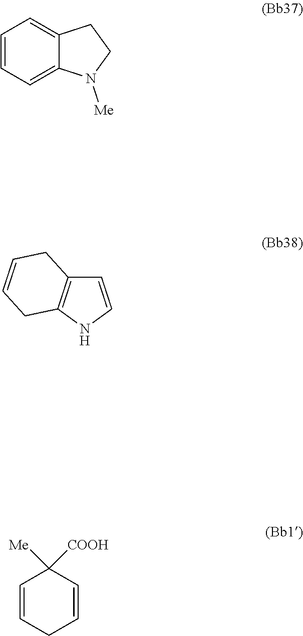 [SMILES: CN1CCc2ccccc21 ; C1=CCc2[nH]ccc2C1 ; CC1(C(=O)O)C=CCC=C1]

### <Mechanochemical Reduction of Benzoic Acid>

### Example B1

A 1.5 mL stainless-steel ball mill jar containing one stainless-steel ball having a diameter of 5 mm was charged under air with 0.25 mmol of benzoic acid (Ba1) as a substrate, lithium wire in an amount of 3.0 equiv relative to the substrate, ethylenediamine in an amount of 6.0 equiv relative to the substrate, and tetrahydrofuran (THE) in an amount of 6.0 equiv relative to the substrate, and the ball mill jar was capped. Thereafter, the ball mill jar was mounted to a ball mill, and a reaction was carried out at room temperature (internal temperature: 25° C.) for 30 minutes by performing shaking and stirring at a vibration frequency of 30 Hz. Then, the ball mill jar was uncapped, and water was added to terminate the reaction, thereby obtaining cyclohexa-2, 5-diene-1-carboxylic acid (Bb1) as a reaction product. The ¹H NMR yield of the reaction product was 38%.

### Examples B2 to B8

A 5 mL stainless-steel ball mill jar containing one stainless-steel ball having a diameter of 10 mm was charged under air with W_{B} mmol of substrate Ba1, lithium wire in an amount of X_{B} equiv relative to the substrate, ethylenediamine in an amount of Y_{B} equiv relative to the substrate, and tetrahydrofuran (THF) in an amount of Z_{B} equiv relative to the substrate, and the ball mill jar was capped. Thereafter, the ball mill jar was mounted to a ball mill, and a reaction was carried out at room temperature (internal temperature: 25° C.) for T_{B} minutes by performing shaking and stirring at a vibration frequency of 30 Hz. Then, the ball mill jar was uncapped, and water was added to terminate the reaction, thereby obtaining cyclohexa-2, 5-diene-1-carboxylic acid (Bb1) as a reaction product. The kinds of W_{B}, X_{B}, Y_{B}, Z_{B}, T_{B}, and Bbn and the ¹H NMR yield of the reaction products are shown in Table 3.

TABLE 3

| | W_{B} | X_{B} | Y_{B} | Z_{B} | T_{B} | ¹H NMR |
|---|---|---|---|---|---|---|
| Example | (equiv) | (equiv) | (equiv) | (equiv) | (min) | yield |
| B1 | 0.25 | 3.0 | 6.0 | 6.0 | 30 | 38% |
| B2 | 0.5 | 3.0 | 6.0 | 6.0 | 30 | 53% |
| B3 | 1.0 | 3.0 | 6.0 | 6.0 | 30 | 94% |
| B4 | 1.0 | 3.0 | 6.0 | 6.0 | 5 | 94% |
| B5 | 1.0 | 3.0 | 6.0 | 6.0 | 1 | 96% |
| B6 | 1.0 | 2.5 | 5.0 | 6.0 | 1 | 96% |
| B7 | 1.0 | 2.0 | 4.0 | 6.0 | 1 | 81% |
| B8 | 1.0 | 2.58 | 5.0 | 0 | 1 | 96% |

### Reference Example B1

A test tube was charged with 1.0 mmol of substrate Ba1, lithium wire in an amount of 2.5 equiv relative to the substrate, and ethylenediamine in an amount of 5.0 equiv relative to the substrate, and a reaction was carried out in a nitrogen atmosphere at room temperature (internal temperature: 25° C.) for 120 minutes. Then, water was added to terminate the reaction, thereby obtaining cyclohexa-2, 5-diene-1-carboxylic acid (Bb1) as a reaction product. The 1H NMR yield of the reaction product was 44%.

<Mechanochemical Reduction of n-Butoxybenzene>

### Example B9

A 5 mL stainless-steel ball mill jar containing one stainless-steel ball having a diameter of 10 mm was charged under air with 1.0 mmol of n-butoxybenzene (Ba2) as a substrate, lithium wire in an amount of 2.5 equiv relative to the substrate, ethylenediamine in an amount of 5.0 equiv relative to the substrate, and tert-butanol in an amount of 1.5 equiv relative to the substrate, and the ball mill jar was capped. Thereafter, the ball mill jar was mounted to a ball mill, and a reaction was carried out at room temperature (internal temperature: 25° C.) for 1 minute by performing shaking and stirring at a vibration frequency of 30 Hz. Then, the ball mill jar was uncapped, and water was added to terminate the reaction, thereby obtaining 1-n-butoxy-1, 4-cyclohexadiene (Bb2), 1-n-butoxy-1-cyclohexene (Bb2′), phenol (Bb2″), and 1-n-butoxy-1, 3-cyclohexadiene (Bb2′″) as reaction products. The conversion of n-butoxybenzene (Ba2) was 85%, the ¹H NMR yield of reaction product Bb2 was 23%, the ¹H NMR yield of reaction product Bb2′ was 6%, the ¹H NMR yield of reaction product Bb2″ was 7%, and the ¹H NMR yield of reaction product Bb2′″ was 32%.

### Example B10

Reaction products were obtained in the same manner as in Example B9 except that tetrahydrofuran (THF) was further charged in an amount of 5.0 equiv. The conversion of n-butoxybenzene (Ba2) was 82%, the ¹H NMR yield of reaction product Bb2 was 57%, the ¹H NMR yield of reaction product Bb2′ was 7%, the ¹H NMR yield of reaction product Bb2″ was 5%, and the ¹H NMR yield of reaction product Bb2″′ was 9%.

### Example B11

Reaction products were obtained in the same manner as in Example B9 except that lithium wire in an amount of 3.0 equiv relative to the substrate, ethylenediamine in an amount of 6.0 equiv relative to the substrate, and tert-butanol in an amount of 2.5 equiv relative to the substrate were charged, and tetrahydrofuran (THF) was further charged in an amount of 5.0 equiv. The conversion of n-butoxybenzene (Ba2) was 100%, the ¹H NMR yield of reaction product Bb2 was 89%, the ¹H NMR yield of reaction product Bb2′ was 11%, the ¹H NMR yield of reaction product Bb2″ was 0%, and the ¹H NMR yield of reaction product Bb2′″ was 0%.

### <Mechanochemical Reduction of Various Substrates>

### Examples B12 to B32

A 5 mL stainless-steel ball mill jar containing one stainless-steel ball having a diameter of 10 mm was charged under air with W_{B} mmol of substrate Ban, X_{B} mmol of lithium wire, Y_{B} mmol of ethylenediamine, and V_{B} mmol of tert-butanol, and the ball mill jar was capped. Thereafter, the ball mill jar was mounted to a ball mill, and a reaction was carried out at room temperature (internal temperature: 25° C.) for T_{B} minutes by performing shaking and stirring at a vibration frequency of 30 Hz. Then, the ball mill jar was uncapped, and water was added to terminate the reaction, thereby obtaining reaction product Bbn.

The kinds of Ban, W_{B}, X_{B}, Y_{B}, V_{B}, T_{B}, and Bbn and the yields (isolated yield and/or ¹H NMR yield) of the reaction products are shown in Table 4.

TABLE 4

| | | | | | | | Reaction | | |
|---|---|---|---|---|---|---|---|---|---|
| | Substrate | W_{B} | X_{B} | Y_{B} | V_{B} | T_{B} | product | Isolated | NMR |
| Example | Ban | (mmol) | (mmol) | (mmol) | (mmol) | (min) | Bbn | yield | yield |
| B12 | Ba1 | 1.00 | 2.50 | 5.00 | | 1 | Bb1 | 86% | 96% |
| B13 | Ba3 | 1.00 | 2.50 | 5.00 | | 1 | Bb3 | 99% | |
| B14 | Ba4 | 1.00 | 2.51 | 5.00 | | 1 | Bb4 | 93% | |
| B15 | Ba5 | 1.00 | 2.53 | 5.00 | | 1 | Bb5 | 99% | |
| B16 | Ba6 | 0.50 | 2.51 | 5.00 | | 1 | Bb6 | | 46% |
| B17 | Ba7 | 1.00 | 3.06 | 6.00 | 1.50 | 1 | Bb7 | | 84% |
| B18 | Ba8 | 1.00 | 3.00 | 6.00 | 1.50 | 1 | Bb8 | 75% | 85% |
| B19 | Ba9 | 1.00 | 3.01 | 6.00 | 1.50 | 1 | Bb9 | 79% | 86% |
| B20 | Ba10 | 1.01 | 2.55 | 5.00 | 1.50 | 1 | Bb10 | 86% | 93% |
| B21 | Ba11 | 1.00 | 2.57 | 5.00 | 1.50 | 1 | Bb11 | 70% | 82% |
| B22 | Ba12 | 1.00 | 2.53 | 5.00 | 1.50 | 1 | Bb12 | 68% | 81% |
| B23 | Ba13 | 1.01 | 3.00 | 6.00 | 1.50 | 1 | Bb13 | 56% | 55% |
| B24 | Ba14 | 0.51 | 2.57 | 5.00 | 1.50 | 1 | Bb14 | 70% | 78% |
| B25 | Ba15 | 1.00 | 5.02 | 10.00 | 3.00 | 2 | Bb15 | | 39% |
| B26 | Ba16 | 1.00 | 3.52 | 7.00 | | 1 | Bb16 | 58% | 72% |
| B27 | Ba17 | 1.00 | 2.97 | 6.00 | | 1 | Bb17 | 70% | 82% |
| B28 | Ba18 | 0.75 | 2.98 | 6.00 | 2.25 | 1 | Bb18 | 39% | 54% |
| B29 | Ba19 | 0.50 | 2.55 | 5.00 | 1.50 | 1 | Bb19 | 54% | 65% |
| B30 | Ba20 | 0.75 | 2.97 | 6.00 | 1.88 | 1 | Bb20 | 73% | 77% |
| B31 | Ba21 | 0.75 | 3.06 | 6.00 | 1.88 | 1 | Bb21 | 79% | 87% |
| B32 | Ba22 | 0.50 | 2.50 | 5.00 | 1.50 | 1 | Bb22 | 95% | |

### <Mechanochemical Reduction of Various Substrates Using Tetrahydrofuran (THF)>

### Examples B33 to B41

A 5 mL stainless-steel ball mill jar containing one stainless-steel ball having a diameter of 10 mm was charged under air with W_{B} mmol of substrate Ban, lithium wire in an amount of X_{B} mmol relative to the substrate, ethylenediamine in an amount of Y_{B} mmol relative to the substrate, tert-butanol in an amount of V_{B} mmol relative to the substrate, and tetrahydrofuran (THF) in an amount of Z_{B} mmol relative to the substrate, and the ball mill jar was capped. Thereafter, the ball mill jar was mounted to a ball mill, and a reaction was carried out at room temperature (internal temperature: 25° C.) for T_{B} minutes by performing shaking and stirring at a vibration frequency of 30 Hz. Then, the ball mill jar was uncapped, and water was added to terminate the reaction, thereby obtaining reaction product Bbn. The kinds of Ban, W_{B}, X_{B}, Y_{B}, V_{B}, Z_{B}, T_{B}, and Bbn and the yields (isolated yield and/or ¹H NMR yield) of the reaction products are shown in Table 5.

TABLE 5

| | | | | | | | | Reaction | | |
|---|---|---|---|---|---|---|---|---|---|---|
| | Substrate | W_{B} | X_{B} | Y_{B} | V_{B} | Z_{B} | T_{B} | product | Isolated | NMR |
| Example | Ban | (mmol) | (mmol) | (mmol) | (mmol) | (mmol) | (min) | Bbn | yield | yield |
| B33 | Ba2 | 1.00 | 3.01 | 6.00 | 2.50 | 5.00 | 1 | Bb2 | 77% | 87% |
| B34 | Ba23 | 1.00 | 3.06 | 6.00 | 2.50 | 5.00 | 1 | Bb23 | 76% | 81% |
| B35 | Ba24 | 0.99 | 3.01 | 6.00 | 2.50 | 5.00 | 1 | Bb24 | | 58% |
| B36 | Ba25 | 1.01 | 3.01 | 6.00 | 2.50 | 5.00 | 1 | Bb25 | | 56% |
| B37 | Ba26 | 1.00 | 3.00 | 6.00 | 2.50 | 5.00 | 1 | Bb26 | | 61% |
| B38 | Ba27 | 1.00 | 3.57 | 7.00 | 2.50 | 5.00 | 1 | Bb27 | 63% | 66% |
| B39 | Ba28 | 1.00 | 3.01 | 6.00 | 2.50 | 5.00 | 1 | Bb28 | 84% | 90% |
| B40 | Ba29 | 1.01 | 3.06 | 6.00 | 1.50 | 5.00 | 1 | Bb29 | 70% | 71% |
| B41 | Ba30 | 0.50 | 2.50 | 5.00 | 1.50 | 2.50 | 1 | Bb30 | 48% | 53% |

<Production of Cyclic γ-Keto Acid by Mechanochemical Reduction of m-Anisic Acid>

### Example B42

A 5 mL stainless-steel ball mill jar containing one stainless-steel ball having a diameter of 10 mm was charged under air with 1.00 mmol of substrate Ba31 (m-anisic acid), 3.06 mmol of lithium wire, and 6.00 mmol of ethylenediamine, and the ball mill jar was capped. Thereafter, the ball mill jar was mounted to a ball mill, and a reaction was carried out at room temperature (internal temperature: 25° C.) for 1 minute by performing shaking and stirring at a vibration frequency of 30 Hz. Then, the ball mill jar was uncapped, and water was added to terminate the reaction, thereby obtaining reaction product Bb31 (5-oxo-2-cyclohexene-1-carboxylic acid). The isolated yield of reaction product Bb31 was 73%, and the ¹H NMR yield was 80%.

### <Mechanochemical Reduction of 1-Naphthoic Acid>

### Example B43

A 5 mL stainless-steel ball mill jar containing one stainless-steel ball having a diameter of 10 mm was charged under air with 1.00 mmol of substrate Ba32 (1-naphthoic acid), 2.97 mmol of lithium wire, and ethylenediamine in an amount of 6.00 mmol relative to the substrate, and the ball mill jar was capped. Thereafter, the ball mill jar was mounted to a ball mill, and a reaction was carried out at room temperature (internal temperature: 25° C.) for 1 minute by performing shaking and stirring at a vibration frequency of 30 Hz. Then, the ball mill jar was uncapped, and water was added to terminate the reaction, thereby obtaining reaction product Bb32 (1,4-dihydronaphthalene-1-carboxylic acid). The isolated yield of reaction product Bb32 was 83%, and the ¹H NMR yield was 96%.

### Example B44

A 5 mL stainless-steel ball mill jar containing one stainless-steel ball having a diameter of 10 mm was charged under air with 0.5 mmol of Ba32 (1-naphthoic acid) as a substrate, 3.06 mmol of lithium wire, 6.00 mmol of ethylenediamine, and 1.50 mmol of tert-butanol, and the ball mill jar was capped. Thereafter, the ball mill jar was mounted to a ball mill, and a reaction was carried out at room temperature (internal temperature: 25° C.) for 1 minute by performing shaking and stirring at a vibration frequency of 30 Hz. Then, the ball mill jar was uncapped, and water was added to terminate the reaction, thereby obtaining a crude reaction mixture. Analysis of the crude reaction mixture revealed that the ¹H NMR yield of reaction product Bb32′ (1, 4, 5, 8-tetrahydro-1-naphthoic acid) was 49%. It was also revealed that 18% (¹H NMR yield) of reaction product Bb32 (1,4-dihydronaphthalene-1-carboxylic acid) and 15% (1H NMR yield) of 1, 2, 3, 4, 5, 8-hexahydronaphthalene-1-carboxylic acid were formed as by-products.

### <Mechanochemical Reduction of 1-Indanol Under Optimized Conditions>

### Example B45

A 5 mL stainless-steel ball mill jar containing one stainless-steel ball having a diameter of 10 mm was charged under air with 0.75 mmol of substrate Ba33 (1-indanol), 3.36 mmol of lithium wire, 6.75 mmol of ethylenediamine, and 2.25 mmol of tert-butanol, and the ball mill jar was capped. Thereafter, the ball mill jar was mounted to a ball mill, and a reaction was carried out at room temperature (internal temperature: 25° C.) for 1 minute by performing shaking and stirring at a vibration frequency of 30 Hz. Then, the ball mill jar was uncapped, and water was added to terminate the reaction, thereby obtaining reaction product Bb33 (2, 3, 4, 7-tetrahydro-1H-indene). The isolated yield of reaction product Bb33 was 51%, and the ¹H NMR yield was 54%.

Reaction product Bb33′ (4,7-dihydro-1-indanol), which could result from reduction of the phenyl ring of substrate Ba33 (1-indanol), was not detected.

### <Mechanochemical Reduction of N-(2-Phenylethyl) Acetamide Under Optimized Conditions>

### Example B46

A 5 mL stainless-steel ball mill jar containing one stainless-steel ball having a diameter of 10 mm was charged under air with 0.50 mmol of substrate Ba34 (N-(2-phenylethyl) acetamide), 2.57 mmol of lithium wire, 5.00 mmol of ethylenediamine, and 1.50 mmol of tert-butanol, and the ball mill jar was capped. Thereafter, the ball mill jar was mounted to a ball mill, and a reaction was carried out at room temperature (internal temperature: 25° C.) for 1 minute by performing shaking and stirring at a vibration frequency of 30 Hz. Then, the ball mill jar was uncapped, and water was added to terminate the reaction, thereby obtaining reaction product Bb34 (1-(2-aminoethyl)-1, 4-cyclohexadiene). The ¹H NMR yield of reaction product Bb33 was 60%.

As a main inseparable by-product, Bb34′ (N-(2-(cyclohexa-1, 4-dien-1-yl)ethyl) acetamide) was formed in 12% ¹H NMR yield.

### <Mechanochemical Reduction of Tetraphenylethylene Using Li>

### Example B47

A 5 mL stainless-steel ball mill jar containing one stainless-steel ball having a diameter of 10 mm was charged under air with 0.50 mmol of substrate Ba35 (tetraphenylethylene; the same as substrate Aa13), 3.01 mmol of lithium wire, and 6.00 mmol of ethylenediamine, and the ball mill jar was capped. Thereafter, the ball mill jar was mounted to a ball mill, and a reaction was carried out at room temperature (internal temperature: 25° C.) for 1 minute by performing shaking and stirring at a vibration frequency of 30 Hz. Then, the ball mill jar was uncapped, and water was added to terminate the reaction, thereby obtaining reaction product Bb35 (1, 1, 2, 2-tetraphenylethane; the same as reaction product Ab13). The isolated yield of reaction product Bb35 was 82%, and the ¹H NMR yield was 91%.

### <Mechanochemical Reduction of Acridine>

### Example B48

A 5 mL stainless-steel ball mill jar containing one stainless-steel ball having a diameter of 10 mm was charged under air with 1.00 mmol of substrate Ba36 (acridine), 3.00 mmol of lithium wire, and 6.00 mmol of ethylenediamine, and the ball mill jar was capped. Thereafter, the ball mill jar was mounted to a ball mill, and a reaction was carried out at room temperature (internal temperature: 25° C.) for 1 minute by performing shaking and stirring at a vibration frequency of 30 Hz. Then, the ball mill jar was uncapped, and water was added to terminate the reaction, thereby obtaining reaction product Bb36 (9, 10-dihydroacridine). The isolated yield of reaction product Bb36 was 97%.

### <Mechanochemical Reduction of N-Methylindole>

### Example B49

A 5 mL stainless-steel ball mill jar containing one stainless-steel ball having a diameter of 10 mm was charged under air with 1.00 mmol of substrate Ba37 (N-methylindole), 3.52 mmol of lithium wire, 7.00 mmol of ethylenediamine, and 5.00 mmol of tetrahydrofuran (THF), and the ball mill jar was capped. Thereafter, the ball mill jar was mounted to a ball mill, and a reaction was carried out at room temperature (internal temperature: 25° C.) for 1 minute by performing shaking and stirring at a vibration frequency of 30 Hz. Then, the ball mill jar was uncapped, and water was added to terminate the reaction, thereby obtaining reaction product Bb37 (1-methyl-2,3-dihydroindole). The isolated yield of reaction product Bb37 was 54%, and the ¹H NMR yield was 60%.

### <Mechanochemical Reduction of Indole>

### Example B50

A 5 mL stainless-steel ball mill jar containing one stainless-steel ball having a diameter of 10 mm was charged under air with 0.75 mmol of substrate Ba38 (indole), 2.91 mmol of lithium wire, 6.00 mmol of ethylenediamine, 3.75 mmol of tetrahydrofuran (THF), and 1.88 mmol of tert-butanol, and the ball mill jar was capped. Thereafter, the ball mill jar was mounted to a ball mill, and a reaction was carried out at room temperature (internal temperature: 25° C.) for 1 minute by performing shaking and stirring at a vibration frequency of 30 Hz. Then, the ball mill jar was uncapped, and water was added to terminate the reaction, thereby obtaining reaction product Bb38 (4,7-dihydro-1H-indole). The isolated yield of reaction product Bb38 was 37%, and the ¹H NMR yield was 48%.

### <Mechanochemical Reduction of Benzoic Acid Using Methyl Iodide>

### Example B51

A 5 mL stainless-steel ball mill jar containing one stainless-steel ball having a diameter of 10 mm was charged under air with 1.00 mmol of substrate Ba1, 2.50 mmol of lithium wire, and 5.00 mmol of ethylenediamine, and the ball mill jar was capped. Thereafter, the ball mill jar was mounted to a ball mill, and a reaction was carried out at room temperature (internal temperature: 25° C.) for 1 minute by performing shaking and stirring at a vibration frequency of 30 Hz. Then, the ball mill jar was uncapped, methyliodide was added in an amount of 3.0 equiv relative to the substrate, and a reaction was carried out at room temperature (internal temperature: 25° C.) for 5 minutes by performing shaking and stirring at a vibration frequency of 30 Hz. Then, the ball mill jar was uncapped, and water was added to terminate the reaction, thereby obtaining reaction product Bb1′ (1-methylcyclohexa-2, 5-diene-1-carboxylic acid). The ¹H NMR yield of the reaction product Bb1′ was 51%.

### <<Mechanochemical Reduction on Gram-Scale>>

Aromatic hydrocarbon compounds Ba5 and Ba36 and reaction products Bb5 and Bb36 used in Examples C1 to C3 are the same compounds as those used in Example B15 and Example B48.

### <Mechanochemical Reduction of 3,5-Dimethylbenzoic Acid on Gram-Scale>

### Example C1

A 10 mL stainless-steel ball mill jar containing two stainless-steel balls having a diameter of 15 mm was charged under air with 7.0 mmol (1.05 g) of substrate Ba5 (3,5-dimethylbenzoic acid), 17.5 mmol (122.0 mg) of lithium wire cut into small pieces of about 3 mm, and 35.0 mmol (2.104 g) of ethylenediamine, and the ball mill jar was capped. Thereafter, the ball mill jar was mounted to a ball mill, and a reaction was carried out at room temperature (internal temperature: 25° C.) for 1 minute by performing shaking and stirring at a vibration frequency of 30 Hz. Then, the ball mill jar was uncapped, and water was added to terminate the reaction. The contents were washed with an organic solvent and washed using an evaporator, thereby obtaining reaction product Bb5 (3,5-dimethyl-2, 5-cyclohexadiene-1-carboxylic acid). The isolated yield of reaction product Bb5 was 98% (6.85 mmol, 1.042 g).

### <Mechanochemical Reduction of Acridine on Gram-Scale>

### Example C2

A 10 mL stainless-steel ball mill jar containing two stainless-steel balls having a diameter of 15 mm was charged under air with 6.0 mmol (1.075 g) of substrate Ba36 (acridine), 18.0 mmol (125.5 mg) of lithium wire cut into small pieces of about 3 mm, and 36.0 mmol (2.164 g) of ethylenediamine, and the ball mill jar was capped. Thereafter, the ball mill jar was mounted to a ball mill, and a reaction was carried out at room temperature (internal temperature: 25° C.) for 1 minute by performing shaking and stirring at a vibration frequency of 30 Hz. Then, the ball mill jar was uncapped, and water was added to terminate the reaction, thereby obtaining reaction product Bb36 (9,10-dihydroacridine). The isolated yield of reaction product Bb36 was 95% (5.72 mmol, 1.037 g).

### Example C3

A 10 mL stainless-steel ball mill jar containing two stainless-steel balls having a diameter of 10 mm was charged under air with 1.073 g (6.0 mmol) of substrate Ba36 (acridine), a metallic sodium lump in an amount of 2.6 equiv relative to the substrate, 1,3-dimethyl-2-imidazolidinone in an amount of 6.0 equiv relative to the substrate, and D-(+)-glucose as a proton source in an amount of 3.0 equiv relative to the substrate, and the ball mill jar was capped. Thereafter, the ball mill jar was mounted to a ball mill, and a reaction was carried out at room temperature (internal temperature: 25° C.) for 15 minutes by performing shaking and stirring at a vibration frequency of 30 Hz. Then, the ball mill jar was uncapped, and water was added to terminate the reaction, thereby obtaining reaction product Bb36 (9,10-dihydroacridine). The isolated yield of reaction product Bb36 was 95% (5.72 mmol, 1.030 g).

### {Mechanochemical Reduction Using Alkaline-Earth Metal}

### <<Mechanochemical Reduction Using Metallic Calcium>>

As metallic calcium used in Example D1, a metal calcium lump about 3 to 5 mm long, 3 to 5 mm wide, and 3 mm thick was used.

### <Mechanochemical Reduction of Naphthalene Using Ca>

### Example D1

A 5 mL stainless-steel ball mill jar containing two stainless-steel balls having a diameter of 10 mm was charged under air with 1.0 mmol of substrate D1 (naphthalene; the same as substrate Ba15), 2.5 mmol of metallic calcium, 5.0 mmol of ethylenediamine, and 3.0 mmol of tert-butanol, and the ball mill jar was capped. Thereafter, the ball mill jar was mounted to a ball mill, and a reaction was carried out at room temperature (internal temperature: 25° C.) for 60 minutes by performing shaking and stirring at a vibration frequency of 30 Hz. Then, the ball mill jar was uncapped, and water was added to terminate the reaction, thereby obtaining a reaction mixture. Analysis of the reaction mixture revealed that the isolated yield of the main reaction product D1 (1, 2, 3, 4-tetrahydronaphthalene) was 31%, and the isolated yield of the side reaction product D1′ (1, 4, 5, 8-tetrahydronaphthalene) was 3%.

1. A reduction method comprising reducing a substrate (A) by mechanochemical treatment using at least a metal (B) and a proton source (C),
wherein the substrate (A) is at least one compound including an unsaturated bond, and
the metal (B) includes at least one selected from an alkali metal or an alkaline-earth metal.

2. The reduction method according to claim 1, wherein the substrate (A) includes at least one selected from the group consisting of an aromatic hydrocarbon compound, an aromatic heterocyclic compound, an internal alkyne-containing compound, and a conjugated alkene compound.

3. The reduction method according to claim 1, wherein the metal (B) includes at least one selected from the group consisting of lithium, sodium, and calcium.

4. The reduction method according to claim 1, wherein the proton source (C) includes a sugar compound.

5. The reduction method according to claim 1, wherein in the reducing by mechanochemical treatment, an organic solvent having a melting point of lower than 30° C. is used in an amount of 1.0 mL or less per mmol of the substrate.

6. The reduction method according to claim 1, wherein the reducing by mechanochemical treatment is carried out under conditions of a temperature of 20° C. or higher and/or a non-inert atmosphere.

7. The reduction method according to claim 2, wherein the metal (B) includes at least one selected from the group consisting of lithium, sodium, and calcium.

8. The reduction method according to claim 2, wherein the proton source (C) includes a sugar compound.

9. The reduction method according to claim 2, wherein in the reducing by mechanochemical treatment, an organic solvent having a melting point of lower than 30° C. is used in an amount of 1.0 mL or less per mmol of the substrate.

10. The reduction method according to claim 2, wherein the reducing by mechanochemical treatment is carried out under conditions of a temperature of 20° C. or higher and/or a non-inert atmosphere.
